(19) United States
(12) Patent Application Publication
(10) Pub. No.: US 2006/0020001 A1
(43) Pub. Date: Jan. 26, 2006

(54) Use of cathepsin k inhibitors for the treatment of glaucoma

(76) Inventors: Allan Shepard, Fort Worth, TX (US); Abbot F. Clark, Arlington, TX (US); Nasreen Jacobson, Fort Worth, TX (US)

Correspondence Address: Teresa J Schultz;Alcon Research, R & D Counsel Q 148, 6201 South Freeway, Fort Worth, TX, 76134-2099, US

(21) Appl. No.: 10/537,052

(22) PCT Filed: Dec. 19, 2003
(86) PCT No.: PCT/US03/40511

§ 371 Date: Jun. 2, 2005

Related U.S. Application Data
(60) Provisional application No. 60/436,126, filed on Dec. 23, 2002.

(51) Int. Cl. A61K 31/426

(52) U.S. Cl. 514/370

(57) ABSTRACT

Compositions containing inhibitors of cathepsin K (CTSK) expression and/or activity are provided. Methods for the treatment of glaucoma using the compositions of the invention are further provided.

### BACKGROUND OF THE INVENTION

1. Field of the Invention

The present invention relates to the field of glaucoma treatment. More specifically, the present invention involves the use of antagonists of cathepsin K (CTSK) activity and/or signaling leading to expression, in order to treat glaucoma or ocular hypertension.

2. Description of the Related Art

The disease state referred to as glaucoma is characterized by a permanent loss of visual function due to irreversible damage to the optic nerve. Morphologically or functionally distinct types of glaucoma are typically characterized by elevated intraocular pressure (IOP), which is considered to be causally related to the pathological course of the disease. Disruption of normal aqueous outflow leading to elevated IOP is integral to glaucoma pathophysiology. Ocular hypertension is a condition wherein IOP is elevated but no apparent loss of visual function has occurred; such patients are considered to be at high risk for the eventual development of the visual loss associated with glaucoma. Some patients with glaucomatous field loss have relatively low IOPs. These so called normotension or low tension glaucoma patients can also benefit from agents that lower and control IOP. Drug therapies that have proven to be effective for the reduction of IOP include both agents that decrease aqueous humor production and agents that increase the outflow facility. Such therapies are, in general, administered by one of two possible routes, topically (direct application to the eye) or orally.

Elevated IOP, found in most glaucoma patients, is a result of morphological and biochemical changes in the trabecular meshwork (TM), an aqueous humor filtering tissue located at the iris-cornea angle of the eye. As glaucoma progresses, there is a loss of TM cells and a buildup of extracellular products which inhibit the normal aqueous humor outflow resulting in IOP elevation. In addition to elevated IOP, ischemia, excitotoxicity and other factors may lead to mechanical distortion of the optic nerve head (ONH) ultimately resulting in ONH cupping and loss of retinal ganglion cells (RGC) and axons. The exact mechanism of this pathological process is currently unknown.

The discrimination of the various forms of glaucoma has progressed since the discovery of the primary open angle glaucoma (POAG) gene MYOC in the GLC1A locus. Over 15 different glaucoma genes have been mapped and seven glaucoma genes identified. Two POAG genes (MYOC and OPTN) have been identified from the six mapped genes (GLC1A-GLC1F). One congenital glaucoma gene (CYP1B1) has been identified from the three mapped genes (GLC3A-GLC3B). Four developmental or syndromic forms of glaucoma (FOXC1, PITX2, LMX1B, PAX6) have been identified and two genes have been mapped for pigmentary dispersion/pigmentary glaucoma.

It is likely that multiple disease mechanisms are at work in the various glaucomas and will require specific therapies tailored to each disorder. For example, a drug that effects the expression of enzymes that degrade the extracellular matrix (ECM) of the ONH may not necessarily prevent RGC death caused by excitotoxicity or neurotrophic factor deficit. On the other hand, many different insults, for example apoptosis of RGCs, may converge at a common point or points that are amenable to drug therapy. Current anti-glaucoma therapy is based on lowering IOP by the use of suppressants of aqueous humor formation, agents that enhance uveoscleral outflow, trabeculoplasty, or trabeculectomy. These current therapies do not directly address the pathological damage to the trabecular meshwork, which continues unabated. It would be advantageous to have a therapeutic product including an agent that directly interferes with the pathogenic process.

Cathepsins are members of the papain family of cysteine proteases. Cathepsin K (CTSK, previously known as cathepsin O or cathepsin O2) is involved in osteoclast bone resorption and bone remodeling. CTSK displays endoprotease activity against fibrinogen, has high elastinolytic and collagenolytic activities and may play an important role in extracellular matrix degradation. CTSK is a lysosomal enzyme with selective expression in osteoclasts and embryo/fetal respiratory and gastrointestinal mucosa. CTSK is highly expressed in human ciliary body epithelial cells (pigmented and non-pigmented), iris, and retinal pigment epithelium but not cornea, lens, or retina and has not been profiled in the trabecular meshwork (Ortega et al. 1997).

The identification of CTSK involvement in glaucoma pathogenesis and the use of expression or activity inhibitors as presented herein has not been previously described.

### SUMMARY OF THE INVENTION

The present invention overcomes drawbacks of the prior art by providing compounds targeted to a specific gene product to protect or rescue patients from the damage caused by glaucoma. Specifically, the present invention is drawn to compositions for methods of treating glaucoma (with or without elevated IOP) and ocular hypertension through the administration of one or more CTSK inhibitors.

The method comprises administering to the subject a therapeutically effective amount of a composition including at least one non-nucleotide or non-protein agent that inhibits expression and/or signaling leading to expression of CTSK, and a pharmaceutically acceptable carrier.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a photographic depiction that illustrates results of differential cDNA subtraction hybridization (FIG. 1A) and Virtual Northern Blot analysis (FIG. 1B) of CTSK in glaucomatous vs. normal TM cell lines.

FIG. 2 is a graph depicting quantitative polymerase chain reaction (PCR) analysis of CTSK expression in pooled normal or glaucomatous TM cell lines.

FIG. 3 is a graph depicting Affymetrix GeneChip U133A analysis of pooled NTM or GTM cell lines.

### DETAILED DESCRIPTION PREFERRED EMBODIMENTS

According to the present invention, the protease CTSK has been identified as being up-regulated in glaucomatous TM cells and tissues. Expression of CTSK under these conditions indicates a causal or effector role for CTSK in glaucoma pathogenesis. It has been found that elevated levels of CTSK may function pathophysiologically by destroying extracellular matrix required for normal filtration and cellular function in the TM. Disruption of normal aqueous outflow leading to elevated IOP is integral to glaucoma pathophysiology. The present invention is directed to the use of antagonists of CTSK in the treatment of glaucoma. The advantage of the present invention is that a specific gene product has been identified to which specific compounds may be targeted to protect or rescue patients from the damage caused by glaucoma. The compositions and methods of the present invention are intended to directly interfere with the pathogenic process.

This invention is directed to the treatment of glaucoma by the inhibition of CTSK. It is contemplated that any CTSK inhibiting compound will be useful in the methods of the present invention. The inventors contemplate that any of the compounds disclosed in U.S. Pat. Nos. 5,830,850; 6,057,362; 5,998,470; and 6,034,077 (all incorporated herein by reference); or described in the literature (Altmann et al. 2002; Billington et al. 2000; Bossard et al. 1999; Bromme et al. 1996; Falgueyret et al. 2001; Fenwick et al. 2001a; Fenwick et al. 2001b; Kamolmatyakul et al. 2001; Katunuma et al. 2000; Katunuma et al. 1999; LaLonde et al. 1998; Lark et al. 2002; Leung-Toung et al. 2002; Marquis et al. 2001a; Marquis et al. 1999; Marquis et al. 2001b; Marquis et al. 1998; Matsumoto et al. 1999; McGrath et al. 1997; Patil et al. 2002a & b; Percival et al. 1999; Schick et al. 1998; Smith et al. 2001; Stroup et al. 2001; Thompson et al. 1997; Thompson et al. 1999; Thompson et al. 1998; Turk et al. 1997; Votta et al. 1997; Yamashita and Dodds 2000; Yamashita et al. 1999; Zhao et al. 1997; all incorporated herein by reference) would be suitable for use in the compositions and methods of the present invention.

More specifically, preferred CTSK antagonists for use in the present invention include, but are not limited to, monensin, brefeldin A, tunicamycin and 1,3-bis(acylamino)-2-propanone derivatives, cathepsin K antisense, triple helix and/or ribozyme molecules, inhibitors of cathepsin K enzymatic activity, azepanone-based inhibitors, cyclic ketones, fluoromethyl ketones, vinyl sulfones, peptide aldehydes, nitriles, α-ketocarbonyl compounds, including α-diketones, α-keto-esters, α-ketoamides, and α-ketoacids, halomethyl ketones, diazomethyl ketones, (acyloxy)-methyl ketones, ketomethylsulfonium salts, epoxy succinyl compounds, cycloaltilisin 6, cycloaltilisin 7, AC-3-1, AC-3-3, AC-5-1, haploscleridamine, 5-(2-morpholin-4-yl-thoxy)-benzofuran-2-carboxylic acid ((S)-3-methyl-1-[3-oxo-1-[2-(3-pyridin-2-yl-phenyl)-ethenoyl]-azepan-4-ylcarbanoyl)-butyl)-amide (SB-331750), SB-357114 (Stroup et al. 2001), peptidomimetic aminomethyl ketones, α,α′-diacylamino ketones, alkoxymethyl ketones, cyanamides, pyridoxal propionate derivatives (including Clik-164 and Clik-166), SB-290190 (Leung-Toung et al. 2002), α-alkoxyketone derivatives, cyanamide derivatives, and arylaminoethyl amides such as N_{α}-acyl-α-amino acid-(arylaminoethyl)amides (Altmann et al. 2002). Additional preferred compounds include:
(2S,1′S)-2-(benzyloxycarbonyl)amino-N-[1′-(2-carboxythiazol-4-yl)-3′-methylbutyl]-4-methylpentanamide;
(2S,1′S)-2-(benzyloxycarbonyl)amino-N-[1′-(2-carboxamidothiazol-4-yl)-3′-methylbutyl]-4-methylpentanamide;
(2S,1′S)-2-(benzyloxycarbonyl)amino-N-[1′-(2-carboethoxythiazol-4-yl)-3′-methylbutyl]-4-methylpentanamide;
(2S,1′S)-2-(benzyloxycarbonyl)amino-N-[1′-(2-cyanothiazol-4-yl)-3′-methylbutyl]-4-methylpentanamide;
(2S,1′S)-2-(benzyoxycarbonyl)amino-N-[1′-[2-(N-benzylcarboxamido)thiazol-4-yl]-3′-methylbutyl]-4-methylpentanamide;
(2S,1′S)-2-(benzyloxycarbonyl)amino-N-[1′-[2-[N′-3-methylpropyl)carboxamido]thiazol-4-yl)]-3′-methylbutyl]-4-methylpentanamide;
(2S,1′S)-2-(benzyloxycarbonyl)amino-N-[1′-[2-[N′-2-phenylethyl) carboxamido]thiazol-4-yl)]-3′-methylbutyl]-4-methylpentanamide;
(2S,1′S)-2-(benzyloxycarbonyl)amino-N-[1′-(4-carboethoxythiazol-2-yl)-3′-methylbutyl]-4-methylpentanamide;
(2S,1′S)-2-(benzyloxycarbonyl)amino-N-[1′-(4-carboxythiazol-2-yl)-3′-methylbutyl]-4-methylpentanamide;
(2S,1′S)-2-(benzyloxycarbonyl)amino-N-[1′-(4-carboethoxythiadiazol-2-yl)-3′-methylbutyl]-4-methylpentanamide;
(2S,1′S)-2-(benzyloxycarbonyl)amino-N-[1′-(2-carbo-2,2,2-trifluoroethoxythiazol-4-yl)-3′-methylbutyl]-4-methylpentanamide;
(2S,1′S)-2-(benzyloxycarbonyl)amino-N-[1′-(4-carboethoxyoxadiazol-2-yl)-3′-methylbutyl]4-methylpentanamide;
(2S,1′S)-2-(benzyloxycarbonyl-L-leucinyl)amino-N-[1′-(4-carboethoxythiazol-2-yl)-3′-methylbutyl]-4-methylpentanamide;
(2S,1′S)-2-(benzyloxycarbonyl)amino-N-[1′-(4-carboxamidooxadiazol-2-yl)-3′-methylbutyl]-4-methylpentanamide;
(2S,1′S)-2-(benzyloxycarbonyl)amino-N-[1′-(2-carboethoxythiazol-4-yl)-3′-methylbutyl]-3-phenylpropanamide;
(2S,1′S)-2-(benzyloxycarbonyl-L-leucinyl)amino-N-[1′-(2-carboethoxythiazol-4-yl)-3′-methylbutyl]-4-methylpentanamide;
(2S,1′S)-2-(benzyloxycarbonyl)amino-N-[1′-(5-mercapto-1,2,4-oxadiazol-3-yl)-3′-methylbutyl]-4-methylpentanamide;
(2S,1′S)-2-(benzyloxycarbonyl)amino-N-[1′-(2-mercaptothiazol-4-yl)-3′-methylbutyl]-4-methylpentanamide;
(2S)-2-(benzyloxycarbonyl)amino-N-(4-carboethoxythiazol-2-yl)methyl-4-methyl pentanamide;
(2S,1′S)-2-(benzyloxycarbonyl)amino-N-[1′-(2-benzyloxycarbonylthiazol-4-yl]-3′-methylbutyl]-4-methylpentanamide;
(2S,1′S)-2-(benzyloxycarbonyl)amino-4-methyl-N-[3′-methyl-1′-(2-phenoxycarbonylthiazol-4-yl)butyl]pentanamide;
(2S,1′S)-2-(benzyloxycarbonyl)amino-4-methyl-N-[3′-methyl-1′-[2-(2-methylpropyloxy carbonyl)thiazol-4-yl]butyl]pentanamide;
(2R,1′S)-2-(benzyloxycarbonyl)amino-N-[1′-(carboethoxythiazol-2-yl)ethyl]4-methylpentanamide;
(2R,1′R)-2-(benzyloxycarbonyl)amino-N-[1′-(4-carbethoxythiazol-2-yl)ethyl]-4-methylpentanamide;
(2S,1′S)-N-[1′-(2-aminothiazol-4-yl)-3′-methylbutyl]-2-(benzyloxycarbonyl)amino-4-methylpentanamide;
(2S,1′S)-2-(benzyloxycarbonyl)amino-N-[1′-(2-carboethoxythiazol-4-yl)-3′-methylbutyl]-4-methylpentanamide;
(2S,1′S)-2-(benzyloxycarbonyl)amino-N-[1′-(4-carboethoxythiazol-2-yl)-3′-methylbutyl]-4-methylpentanamide;
(2S,1′S)-2-(benzyloxycarbonyl-L-leucinyl)amino-N-[1′-(4-carboethoxythiazol-2-yl)-3′-methylbutyl]-4-methylpentanamide;
(1S)-N-[4-[(1-benzyloxycarbonylamino)-3-methylbutyl]thiazol-2-ylcarbonyl]-N′-(N-benzyloxycarbonyl-L-leucinyl)hydrazide;
N-benzyloxycarbonyl-L-leucinyl-N′-benzyloxycarbonyl-L-leucinyl-L-leucinylhydrazide;
(1S)-N-[2-[(1-benzyloxycarbonylamino)-3-methylbutyl]thiazol-4-ylcarbonyl]-N′-(N-benzyloxycarbonyl-L-leucinyl)hydrazide;
2,2′-(N,N′-bis-benzyloxycarbonyl-L-leucinyl)carbohydrazide;
2,2′-(N,N′-bis-cyclohexylacetyl)carbohydrazide;
2,2′-(N,N′-bis-4-methylpentanoyl)carbohydrazide;
2,2′-(N,N′-bis-cyclopentylacetyl)carbohydrazide;
2,2′-(N,N′-bis-benzyloxycarbonylglycinyl)carbohydrazide;
2,2′-(N,N′-bis-acetyl-L-leucinyl)carbohydrazide;
2,2′-(N,N′-bis-benzyloxycarbonyl-L-alanyl)carbohydrazide;
2-(N-benzyloxycarbonyl-L-leucinyl)-2′-[N′-(4-methylpentanoyl)]carbohydrazide;
2,2′-(N,N′-bis-benzyloxycarbonyl-L-leucinyl)carbohydrazide;
bis-(Cbz-leucinyl)-1,3-diamino-propan-2-one;
bis-1,3-(4-phenoxy-benzoyl)-diamino-propan-2-one;
1-(Cbz-leucinyl)-amino-3-(acetyl-leucinyl)-amino-propan-2-one;
1-(Cbz-leucinyl)-amino-3-(Cbz-glutamyl-t-butyl ester)-amino-propan-2-one;
1-(Cbz-leucinyl)-amino-3-(Cbz-glutamyl)-amino-propan-2-one;
bis-1,3-(Cbz-leucinyl)-diamino-(S)-butanone-2-one;
1-(Cbz-leucinyl)-amino-3-(Cbz-phenylalanyl)-amino-propan-2-one;
1-(Cbz-leucinyl)-amino-3-(Cbz-norleucinyl)-amino-propan-2-one;
1-(Cbz-leucinyl)-amino-3-(Cbz-norvalinyl)-amino-propan-2-one;
bis-1,3-(Cbz-leucinyl)-diamino-5-methyl-(S)-hexan-2-one;
1-(acetyl-leucinyl)-amino-3-(4-phenoxy-benzoyl)-amino-propan-2-one;
1-(Cbz-homo-leucinyl)-amino-(Cbz-leucinyl)-3-amino-propan-2-one;
1-(Cbz-leucinyl)-amino-3-(acetyl-leucinyl)-amino-propan-2-one;
bis-1,3-(4-(3-chloro-2-cyano-phenoxy)-phenyl sulfonamido)-propan-2-one;
bis-1,3-(4-phenoxy-phenyl sulfonamido)-propan-2-one;
1-(Cbz-leucinyl)-amino-3-(4-(3-chloro-2-cyano-phenoxy)-phenyl sulfonamido)-propan-2-one;
1-(Cbz-leucinyl)-amino-3-(tosyl-amino)-propan-2-one;
1-(Cbz-leucinyl)-amino-3-((4-phenoxy-phenyl)-sulfonamido)-propan-2-one;
1-(Cbz-leucinyl)-amino-3-(2-dibenzofuransulfonamido)-propan-2-one;
1-(Cbz-homo-leucinyl)-amino-3-(2-dibenzofuransulfonamido)-propan-2-one;
1-(Cbz-leucinyl)-amino-3-(2-dibenzofuransulfonamido)-(S)-butan-2-one;
1-(Cbz-leucinyl)-amino-3-((4-phenoxy-phenyl)-sulfonamido)-propan-2-one;
1-(Cbz-leucinyl)-amino-3-(2-dibenzofuransulfonamido)-propan-2-one;
1-(Cbz-leucinyl)-amino-3-(2-dibenzofuransulfonamido)-(S)-butan-2-one;
(S)-Phenylmethyl [1-[[[3-[benzyloxycarbonyl-leucinyl-amino]-2-oxopropyl]-1-(benzyl)amino]carbonyl]-3-methylbutyl]carbamate;
(S)-Phenylmethyl [1-[[[3-[(2-dibenzofuranylsulfonyl)amino]-2-oxopropyl]-3-(benzyl)amino]carbonyl]-3-methylbutyl]carbamate;
(S)-Phenylmethyl [1-[[[3-[(2-dibenzofuranylsulfonyl)amino]-2-oxopropyl]-3-(4-pyridinylmethyl)amino]carbonyl]-3-methylbutyl]carbamate;
1-[[3-[(2-dibenzofuranylsulfonyl)amino]-2-oxopropyl]-3-(4-pyridinylmethyl)]benzamide;
(S)-Phenylmethyl [1-[[[3-[(2-dibenzofuranylsulfonyl)amino]-2-oxopropyl]-1-(4-pyridinylmethyl)amino]carbonyl]-3-methylbutyl]carbamate;
(S)-Phenylmethyl [1-[[[3-[(2-dibenzofuranylsulfonyl)amino]-2-oxopropyl]-1-(4-pyridinylmethyl)amino]carbonyl]-3-methylbutyl]carbamate;
2-[N-(N-benzyloxycarbonyl-L-leucinyl)]-2′-[N′-(4-phenoxyphenylsulfonyl)]carbohydrazide;
2-[N-(N-benzyloxycarbonyl-L-alanyl)]-2′-[N-(N-benzyloxycarbonyl-L-leucinyl)]carbohydrazide;
2-[N-(N-benzyloxycarbonyl-L-leucinyl)]-2′-[N′-(4-phenylbenzoyl)]carbohydrazide;
2-[N-(N-benzyloxycarbonyl-L-leucinyl)]-2′-[N′4-methoxybenzoyl)]carbohydrazide;
2-[N-(N-benzyloxycarbonyl-L-leucinyl)]-2′-[N′-(4-phenoxybenzoyl)]carbohydrazide;
2-(N-acetyl)-2′-[N′-(N-benzyloxycarbonyl-L-leucinyl)]carbohydrazide;
2-[N-(N-acetyl-L-leucinyl)]-2′-[N′-(N-benzyloxycarbonyl-L-alanyl)]carbohydrazide;
2-[N-(N-acetyl-L-alanyl)]-2′-[N′-(N-benzyloxycarbonyl-L-leucinyl)]carbohydrazide;
2-[N-(N-benzyloxycarbonyl-L-leucinyl)]-2′-[N′-[4-N,N-dimethylaminomethyl) benzoyl)]]carbohydrazide;
2-[N-(N-benzyloxycarbonyl-L-leucinyl)]-2′-[N′-[4-hydroxy-[3-(4-morpholinomethyl)]]benzoyl]carbohydrazide;
2-[N-N-benzyloxycarbonyl-L-leucinyl)]-2′-[N′-[4-[N,N-dimethylaminomethyl) benzyloxy]carbonyl-L-leucinyl]carbohydrazide;
2-(N-benzoyl)-2′-[N′-(N-benzyloxycarbonyl-L-leucinyl)]carbohydrazide;
2-[N-(N-benzyloxycarbonyl-L-leucinyl)]-2′-[N′-[3-(4-morpholinomethyl)benzoyl]]carbohydrazide;
2-[N-(3-benzyloxybenzoyl)]-2′-[N′-(N-benzyloxycarbonyl-L-leucinyl)]carbohydrazide;
2-[N-(N-benzyloxycarbonyl-L-leucinyl)]-2′-[N′-[4-[3-N-N-dimethylamino)-1-propyloxy]benzoyl]]carbohydrazide;
2-[N-(2-benzyloxybenzoyl)]-2′-[N′-(N-benzyloxycarbonyl-L-leucinyl)]carbohydrazide;
2-[N-(N-benzyloxycarbonyl-L-leucinyl)]-2′-[N′-[3-(4-pyridylmethoxy)benzoyl]]carbohydrazide;
2-[N-(4-benzyloxybenzoyl)]-2′-[N′-(N-benzyloxycarbonyl-L-leucinyl)]carbohydrazide;
2-[N-(N-benzyloxycarbonyl-L-leucinyl)]-2′-[N′-(3-benzyloxy-5-methoxy)benzoyl]carbohydrazide;
2-[N-(N-benzyloxycarbonyl-L-leucinyl)]-2′-[N′-(3-benzyloxy-4,5-dimethoxy) benzoyl]carbohydrazide;
2-[N-(N-benzyloxycarbonyl-L-leucinyl)]-2′-[N′-(3-benzyloxy-5-ethoxy) benzoyl]carbohydrazide;
2-[N-(N-benzyloxycarbonylglycinyl)]-2′-[N′-(N-benzyloxycarbonyl-L-leucinyl)]carbohydrazide;
2-[N-(3-benzyloxybenzoyl)]-2′-[N′-(N-benzyloxycarbonyl-L-prolinyl)]carbohydrazide;
2-[N-(N-benzyloxycarbonyl-L-leucinyl)]-2′-[N′-(4-phenylphenylacetyl)]carbohydrazide;
(2′S)-2-[N-(3-benzyloxybenzoyl)]-2′-[N′-(N-benzyloxycarbonyl-2-aminobutyryl)]carbohydrazide;
2,2′-[N,N′-[bis-4-phenylphenylacetyl)]]carbohydrazide;
(2′RS)-2-[N-(N-benzyloxycarbonyl-L-leucinyl)]-2′-[2-(4-phenylphenoxy)propionyl]carbohydrazide;
2-[N-(3-benzyloxybenzoyl)]-2′-[N′-(4-methylpentanoyl)]carbohydrazide;
(2RS,2′RS)-2,2′-[N,N′-[bis-[2-(4-phenylphenyl)-4-methylpentanoyl)]]]carbohydrazide;
(2′RS)-2-[N-(N-benzyloxycarbonyl-L-leucinyl)]-2′-[N′-[2-(4-phenylphenyl)-4-methylpentanoyl)]]carbohydrazide;
(2′RS)-2-[N-(3-benzyloxybenzoyl)]-2′-[N-[2-(4-phenylphenyl)-4-methylpentanoyl)]]carbohydrazide;
2-[N-(3-benzyloxybenzoyl)]-2′-[N′-(N-benzyloxycarbonyl-N-methyl-L-leucinyl)]carbohydrazide;
2-[N-(3-benzyloxybenzoyl)]-2′-[N′-[N-(2-pyridinylmethoxycarbonyl)-L-leucinyl]]carbohydrazide;
2-[N-[3-(4-pyridylmethoxy)benzoyl]]-2′-[N′-[N-(2-pyridinylmethoxycarbonyl)-L-leucinyl]]carbohydrazide;
(2RS)-2-[N-[2-(4-phenylphenyl)-4-methylpentanoyl)]]-2′-[N′-[N-(2-pyridinylmethoxy carbonyl)-L-leucinyl]]carbohydrazide;
2-[N-(N-benzyloxycarbonyl-L-leucinyl)]-2′-[N-[2-4-phenylphenyl)-4-methylpentanoyl)]]carbohydrazide;
2-[N-(N-benzyloxycarbonyl-L-leucinyl)]-2′-[N′-[2-(4-phenylphenyl)-4-methylpentanoyl)]]carbohydrazide;
2-[N-(N-benzyloxycarbonyl-L-leucinyl)]-2′-[N′-[N-(4-phenylphenyl)-N-(2-methylpropyl) carbamoyl]]carbohydrazide;
2-[N-(3-benzyloxybenzoyl)]-2′-[N′-(N-methyl-L-leucinyl)]carbohydrazide;
2-[N-(N-benzyloxycarbonyl-L-leucinyl)]-2′-[N′-(N-methyl-L-leucinyl)]carbohydrazide;
(1S)-N-[2-[(1-benzyloxycarbonylamino)-3-methylbutyl]thiazol-4-ylcarbonyl]-N′-(4-phenoxyphenylsulfonyl)hydrazide;
(1S)-N-[4-[1-(N-benzyloxycarbonyl-L-leucinylamino)-3-methylbutyl]thiazol-2-ylcarbonyl]-N′-(N-benzyloxycarbonyl-L-leucinyl)hydrazide;
(1S)-N-[2-[(1-benzyloxycarbonylamino)-3-methylbutyl]thiazol-4-ylcarbonyl]-N′-(4-phenylphenylacetyl)hydrazide;
(1S)-N-[2-[(1-benzyloxycarbonylamino)-3-methylbutyl]thiazol-4-ylcarbonyl]-N′-[3-(4-pryidinylmethoxy)benzoyl]hydrazide;
N-[2-(2-chlorophenoxymethyl)thiazol-4-ylcarbonyl]-N′-[N-(4-pyridinylmethoxycarbonyl)-L-leucinyl]hydrazide;
N-[N-(4-pyridinylmethoxycarbonyl)-L-leucinyl]-N′-[2-[4-(1,2,3-thiadiazol-4-yl)phenyl]thiazol-4-ylcarbonyl]hydrazide;
N-[2-[3-(4-chlorophenylsulfonylmethyl)thien-2-yl]thiazol-4-ylcarbonyl]-N′-[N-(4-pyridinylmethoxycarbonyl)-L-leucinyl]hydrazide;
(1S,2′RS)-N-[2-[(1-benzyloxycarbonylamino)-3-methylbutyl]thiazol-4-ylcarbonyl]-N′-[2′-(4-phenylphenylacetyl)-4-methylpentanoyl-]hydrazide;
N-[2-(3-benzyloxyphenyl)thiazol-4-ylcarbonyl]-N′-[N-(2-pyridinylmethoxycarbonyl)-L-leucinyl]hydrazide;
(1RS)-N-[2-[1-(4-phenylphenyl)-3-methylbutyl]thiazolylcarbonyl]-N′-[N-(4-pyridinylmethoxycarbonyl)-L-leucinyl]hydrazide;
N-[2-(2-benzyloxyphenyl)thiazol-4-ylcarbonyl]-N′-[N-(4-pyridinylmethoxycarbonyl)-L-leucinyl]hydrazide;
N-[2-[N-methyl-N-(4-phenylphenyl)amino]thiazol-4-ylcarbonyl]-N′-[N-(4-pyridinyl methoxycarbonyl)-L-leucinyl]hydrazide;
N-(N-benzyloxycarbonyl-L-leucinyl)-N′-[2-(4-phenylbenzyl)thiazol-4-ylcarbonyl]hydrazide;
N-[2-(4-phenylphenylbenzyl)thiazol-4-ylcarbonyl]-N′-[N-(4-pyridinylmethoxycarbonyl)-L-leucinyl]hydrazide;
N-(N-benzyloxycarbonyl-L-leucinyl)-N′-[2-[N-(2-methylpropyl)-N-phenylamino]thiazol-4-ylcarbonyl]hydrazide;
N-[2-[N-(2-methylpropyl)-N-phenylamino]thiazol-4-ylcarbonyl]-N′-N-(4-pyridinyl methoxycarbonyl)-L-leucinyl]hydrazide;
N-[2-(2-benzyloxyphenyl)thiazol-4-ylcarbonyl]-N′-[N-(3-pyridinylmethoxycarbonyl)-L-leucinyl]hydrazide;
N-[2-(2-benzyloxyphenyl)thiazol-4-ylcarbonyl]-N′-[N-(2-pyridinylmethoxycarbonyl)-L-leucinyl]hydrazide;
N-(N-benzyloxycarbonyl-N-methyl-L-leucinyl)-N′-[2-(2-benzyloxyphenyl)thiazol-4-ylcarbonyl]hydrazide;
N-[2-[N-(2-methylpropyl)-N-phenylamino]thiazol-4-ylcarbonyl]-N′-[N-(2-pyridinyl methoxycarbonyl)-L-leucinyl]hydrazide;
N-[2-[N-(2-methylpropyl)-N-phenylamino]thiazol-4-ylcarbonyl]-N′-[N-(3-pyridinyl methoxycarbonyl)-L-leucinyl]hydrazide;
N-[2-(2-methoxyphenyl)thiazol-4-ylcarbonyl]-N′-[N-(4-pyridinylmethoxycarbonyl)-L-leucinyl]hydrazide;
2-[N-(N-benzyloxycarbonyl-L-leucinyl)]-2′-[N′-[4-(N,N-dimethylaminomethyl) benzyloxy]carbonyl-L-leucinyl]carbonhydrazide;
(3S,4S)-3-(2S-2-benzyloxycarbonylamino-2-cyclohexyl-methyl-acetamido)-4-acetoxy-azetidin-2-one;
(3S,4S)-3-{2S-2-(3-phenylpropionoyl)amino-2-cyclo-hexylmethyl-acetamido}-4-acetoxy-azetidin-2-one;
(3S,4S)-3-{2S-2-(3-phenylpropionoyl)amino-2-cyclo-hexylmethyl-acetamido}-4-{4-(2S-2-amino-2-carboxy-ethyl)-phenoxy}-azetidin-2-one;
(3S,4R)-3-{2S-2-(3-phenylpropionoyl)amino-2-cyclo-hexylmethyl-acetamido}-4-{4-(2S-2-amino-2-carboxy-ethyl)-phenoxy}-azetidin-2-one;
(3S,4SR)-3-{2S-2-(3-phenylpropionoyl)amino-2-cyclo-hexylmethyl-acetamido}4-phenylthio-azetidin-2-one;
(3S,4SR)-3-{2S-2-(3-phenylpropionoyl)amino-2-cyclo-hexylmethyl-acetamido}-4-phenylsulfonyl-azetidin-2-one;
(3S,4S)-3-{2S-2-(benzylaminocarbonyl)amino-2-cyclo-hexylmethyl-acetamido}-4-acetoxy-azetidin-2-one;
(3S,4S)-3-{2S-2-(phenylethenylsulfonyl)amino-2-cyclohexylmethyl-acetamido}-4-acetoxy-azetidin-2-one;
(3S,4S)-3-(2S-2-benzyloxycarbonylamino-2-cyclohexylmethyl-acetamido)-4-(3-methyl-phenoxy)-azetidin-2-one;
(3S,4R)-3-(2S-2-benzyloxycarbonyl amino-2-cyclohexymethyl-acetamido)-4-(3-methyl-phenoxy)-azetidin-2-one;
(3S,4S)-3-{2S-2-[3-(pyridin-4-yl) propenoyl]amino-2-cyclohexylmethyl-acetamido}-4-phenoxy-azetidin-2-one; and
(3S,4S)-3-{2S-2-[3-(pyridin-3-yl)propenoyl]amino-2-cyclohexylmethyl-acetamido}4-phenoxy-azetidin-2-one.

Additional inhibitors of CTSK may be identified by one skilled in the art by using enzyme assays with a known small peptide fluorogenic substrate for CTSK. A specific CTSK substrate used may be the cleavable fluorogenic peptide Z-Phe-Arg-AMC (phenylalanine-arginine-aminomethylcoumarin; Bachem Biosciences, Inc., King of Prussia, Pa.) (Votta, Levy et al. 1997). Hydrolysis of this peptide by CTSK may be followed by changes in fluorescence versus time in the presence or absence of selected CTSK inhibitors.

The inventors are unaware of any previous teaching of the use of these compounds for lowering and controlling normal or elevated intraocular pressure (IOP) and treating glaucoma

While bound by no therories, the fundamental principle behind using CTSK antagonists in the treatment of glaucoma is that elevated levels of this enzyme may function pathophysiologically by destroying extracellular matrix required for normal filtration and cellular function in the trabecular meshwork (TM). CTSK antagonists may be administered systemically either orally or intravenously or specifically to the eye via topical or intravitreal injection. The compounds that are useful as CTSK antagonists in the methods of this invention (Compounds) include any and all compounds that inhibit or antagonize CTSK. Such Compounds can be incorporated into various types of ophthalmic formulations for delivery to the eye (e.g., topically, intracamerally, or via an implant). The Compounds are preferably incorporated into topical ophthalmic formulations for delivery to the eye. The Compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a Compound in a physiologically acceptable isotonic aqueous buffer. Further, the ophthalmic solution may include an ophthalmologically acceptable surfactant to assist in dissolving the Compound. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthamic ointment formulations, the active ingredient is combined with a preservative in an appropriate vehicle, such as, mineral oil, liquid lanolin, or white petrolatum. Sterile ophthalmic gel formulations may be prepared by suspending the Compound in a hydrophilic base prepared from the combination of, for example, carbopol-974, (BF Goodrich, Charlotte, N.C.) or the like, according to the published formulations for analogous ophthalmic preparations; preservatives and tonicity agents can be incorporated.

The Compounds are preferably formulated as topical ophthalmic suspensions or solutions, with a pH of about 4 to 8. The establishment of a specific dosage regimen for each individual is left to the discretion of the clinicians. The Compounds will normally be contained in these formulations in an amount 0.01% to 5% by weight, but preferably in an amount of 0.05% to 2% and most preferably in an amount 0.1 to 1.0% by weight. The dosage form may be a solution, suspension, or microemulsion. Thus, for topical presentation 1 to 2 drops of these formulations would be delivered to the surface of the eye 1 to 4 times per day according to the discretion of a skilled clinician.

The Compounds can also be used in combination with other agents for treating glaucoma, such as, but not limited to, β-blockers (preferably timolol, betaxolol, or levobetaxolol), prostaglandins and analogues thereof (preferably travoprost, latanoprost or bimatoprost), carbonic anhydrase inhibitors (preferably acetazolamide or dorzolamide), α₂ agonists (preferably aproclonidine or brimonidine), miotics, and neuroprotectants.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### cDNA Subtraction Screen & Virtual Northern Blot Analysis

CTSK was originally identified in a custom PCR-Select cDNA subtraction screen (Clontech, Palo Alto, Calif.) as being more abundant in glaucomatous than normal TM cells. Human TM cells were derived from donor eyes (Central Florida Lions Eye and Tissue Bank, Tampa, Fla.) and cultured as previously described (Steely, Browder et al. 1992; Wilson, McCartney et al. 1993; Clark, Wilson et al. 1994; Dickerson, Steely et al. 1998; Wang, McNatt et al. 2001).

The cDNA subtraction procedure was essentially performed as follows. Total RNA (700 μg) was isolated from pooled normal (NTM10C, NTM69C, NTM96, NTM57C, NTM53A, NTM95, and NTM93) or glaucomatous TM cell lines (GTM999, GTM59B, GTM19, GTM62, GTM29, and GTM86) as described by Shepard et al. (2001). Poly A+ RNA was subsequently isolated from the total RNA by two rounds of selection with oligo-dT latex beads using a Nucleotrap mRNA Midi kit (Clontech, Palo Alto, Calif.). PCR-Select cDNA subtraction was then performed according to the manufacturers instructions (Clontech, Palo Alto, Calif.).

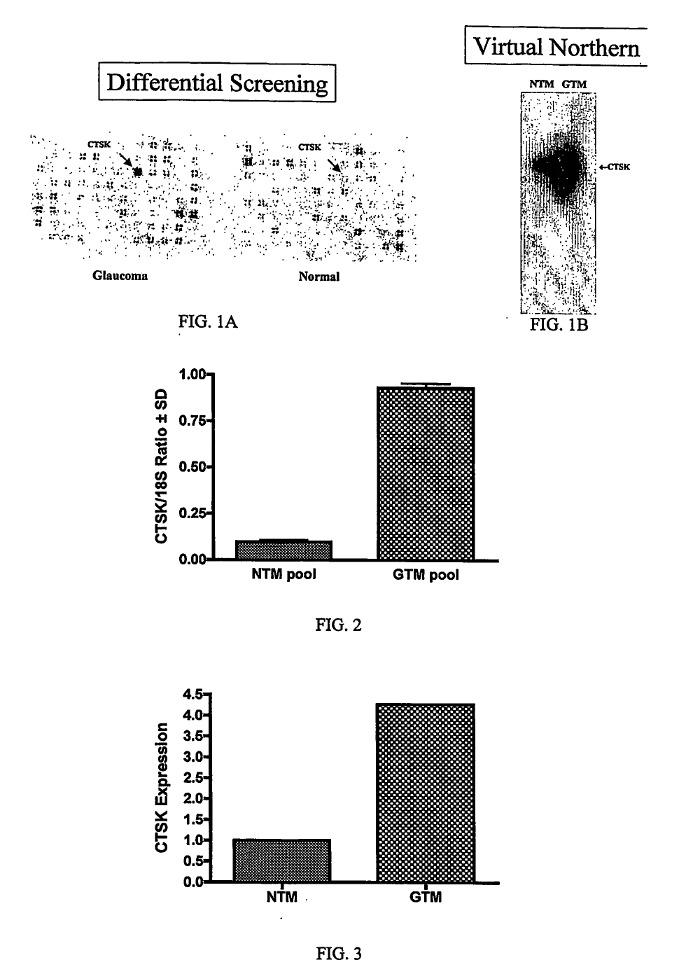

Screening of the cDNA subtraction libraries was performed by PCR-Select Differential Screening according to the manufacturers instructions (Clontech, Palo Alto, Calif.). Five x 96-well plates of subtracted cDNA clones were subjected to differenteial screening analysis (FIG. 1A). Differentially expressed cDNA clones were ordered by relative abundance and CTSK was identified as the most abundant clone. CTSK was also confirmed by Virtual Northern blot analysis (Clontech, Palo Alto, Calif.) as being more abundant in the normal-subtracted glaucoma cDNA library (FIG. 1B). TM cell lines used for pooling for Virtual Northern blot analysis were identical to those used in the cDNA subtraction analysis.

### EXAMPLE 2

### Quantitative PCR

Additional verification of differential expression of CTSK was performed by Quantitative Real-Time PCR (QPCR). First strand cDNA was generated from 1 μg of total RNA isolated from pooled normal or glaucoma TM cell lines (identical to those used in the cDNA Subtraction analysis) using random hexamers and Taqman Reverse Transcription reagents according to the manufacturer's instructions (Applied Biosystems, Foster City, Calif.).

Measurement of CTSK gene expression by QPCR was performed using an ABI Prism 7700 Sequence Detection System (Applied Biosystems, Foster City, Calif.) essentially as described (Shepard et al. 2001). Primers for CTSK QPCR amplification were based on the sequence information in GenBank accession # NM_{—}000396 and were designed using Primer Express software (Applied Biosystems, Foster City, Calif.). Forward and reverse primer sequences were CATATGTGGGACAGGAAGAGAGTTG (nucleotides 734-758) and GGATCTCTCTGTACCCTCTGCATT (nucleotides 788-811), and the TaqMan (Applied Biosystems, Foster City, Calif.) probe sequence was AGCTGCCTTGCCTGTTGGGTTGTACA (nucleotides 761-786) with 6FAM and TAMRA attached to the 5′ and 3′ ends of the probe sequence, respectively. Amplification of the 78-bp CTSK amplicon was normalized to 18S ribosomal RNA levels in each sample using pre-developed 18S rRNA primer/probe set (20×18S Master Mix; Applied Biosystems, Foster City, Calif.). CTSK QPCR consisted of 1× TaqMan Universal Mix (Applied Biosystems, Foster City, Calif.), 0.25×18S rRNA primer/probe set, 900 nM each CTSK primers, 100 nM CTSK TaqMan probe, and 2.5 ng cDNA in a final volume of 25 μl. Thermal cycling conditions consisted of 50° C., 2 min, 95° C. 10 min followed by 40 cycles at 95° C., 15 sec, 60° C., 1 min. Quantitation of relative cDNA concentrations was done using the relative standard curve method as described in PE Biosystems User Bulletin #2 (Applied Biosystems, Foster City, Calif.). Pooled glaucomatous TM cell line cDNA (identical to that used for the cDNA subtraction analysis) was also used for generating the relative standard curve. Data analysis was performed with SDS software version 1.91 (Applied Biosystems, Foster City, Calif.) and MS Excel 97 (Microsoft, Redmond, Wash.). QPCR data are presented as mean±SEM of the CTSK/18S normalized ratio.

### EXAMPLE 3

### Affymetrix GeneChip Analysis

In addition to the identification of CTSK by cDNA subtraction analysis, CTSK was subsequently identified as upregulated in GTM cells by Affymetrix GeneChip (Affymetrix, Santa Clara, Calif.) analysis using pooled normal (NTM94, NTM68B, NTM79B, and NTM55C) or glaucomatous TM cells (GTM19A, GTM54A, GTM62E&G, and SGTM152) (NTM=normal trabecular meshwork; GTM=glaucomatous trabecular meshwork). Essentially, total RNA was collected from the cell lines using TRIZOL reagent according to the manufacturers instructions (Invitrogen, Carlsbad, Calif.), pooled, and subjected to reverse transcription, in vitro transcription, and biotin-labeling of amplified cRNA according to standard Affymetrix protocols (Affymetrix, Santa Clara, Calif.). The Affymetrix Human Genome U133A/B GeneChip (Affymetrix, Santa Clara, Calif.) set was probed with labeled cRNA from either the normal or glaucoma TM cells. Hybridized GeneChips were scanned with a GeneArray scanner (Agilent Technologies, Palo Alto, Calif.). Data was collected and analayzed using Microarray Suite software (Affymetrix, Santa Clara, Calif.).

Subsequent data analysis was done with GeneSpring software (Silicon Genetics, Redwood City, Calif.). For each experiment, data were normalized per chip by dividing each measurement by the 50^{th} percentile of all signal intensity measurements for that chip. The expression ratio for each gene was calculated by dividing the normalized signal per gene in the treated or diseased sample by the median for that gene in the control sample for each experiment. Genes were selected for an expression level above the statistical background by using the Cross-Gene Error Model and setting the baseline equal to the unique base/proportional value for each experiment. Only genes that were flagged as present/marginal on the Affymetrix U133A GeneChip in all experimental conditions were considered for analysis. RNA sources for this study were derived from normal TM cell lines NTM94, NTM68B, NTM79B, and NTM55C and glaucoma TM cell lines GTM19A, GTM54A, GTM62E&G, and SGTM152. CTSK (GenBank #NM_{—}000396) is represented on the U133A GeneChip as probe set 202450_s_at. CTSK was detected at levels 4.3-fold higher in glaucomatous than normal TM cells (FIG. 3).

### EXAMPLE 4

| | Amount in weight % |
|---|---|
| Cathepsin K inhibitor | 0.01-5; 0.05-2; 0.1-1.0 |
| Hydroxypropylmethylcellulose | 0.5 |
| Sodium Chloride | 0.8 |
| Benzalkonium Chloride | 0.01 |
| EDTA | 0.01 |
| NaOH/HCl | q.s. pH 7.4 |
| Purified water | q.s. 100 mL |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents
U.S. Pat. No. 5,830,850
U.S. Pat. No. 5,998,470
U.S. Pat. No. 6,034,077
U.S. Pat. No. 6,057,362

Other Publications
Altmann, E., Renaud, J., Green, J., Farley, D., Cutting, B., Jahnke, W., “Arylaminoethyl amides as novel non-covalent cathepsin K inhibitors,” J. MED. CHEM. 45:2352-4 (2002).
Billington, C. J., Mason, P., Magny, M. C., and Mort, J. S., “The slow-binding inhibition of cathepsin K by its propeptide,” BIOCHEM. BIOPHYS. RES. COMMUN. 276:924-929 (2000).
Bossard, M. J., Tomaszek, T. A., Levy, M. A., Ijames, C. F., Huddleston, M. J., Briand, J., Thompson, S., Halpert, S., Veber, D. F., Carr, S. A., “Mechanism of inhibition of cathepsin K by potent, selective 1,5-diacylcarbohydrazides: a new class of mechanism-based inhibitors of thiol proteases,” BIOCHEMISTRY 38:15893-15902 (1999).
Bromme, D., Klaus, J. L., Okamoto, K., Rasnick, D., Palmer, J. T., “Peptidyl vinyl sulphones: a new class of potent and selective cysteine protease inhibitors: S2P2 specificity of human cathepsin O2 in comparison with cathepsins S and L,” BIOCHEM. J. 315:85-89 (1996).
Clark, A. F., Wilson, K., McCartney, M. D., Miggans, S. T., Kunkle, M., Howe, W., “Glucocorticoid-induced formation of cross-linked actin networks in cultured human trabecular meshwork cells,” IOVS 35:281 (1994).
Dickerson, J. E., Jr., English-Wright, S. L., Clark, A. F., “The effect of dexamethasone on integrin and laminin expression in cultured human trabecular meshwork cells,” EXP EYE RES 66:731 (1998).
Falgueyret, J. P., Oballa, R. M., Okamoto, O., Wesolowski, G., Aubin, Y., Rydzewski, R. M., Prasit, P., Riendeau, D., Rodan, S. B., Percival, M. D., “Novel, nonpeptidic cyanamides as potent and reversible inhibitors of human cathepsins K and L,” J. MED. CHEM. 44:94-104 (2001).
Fenwick, A. E., Garnier, B., Gribble, A. D., Ife, R. J., Rawlings, A. D., Witherington, J., “Solid-phase synthesis of cyclic alkoxyketones, inhibitors of the cysteine protease cathepsin K,” BIOORG. MED. CHEM. LETT. 11:195-198 (2001a).
Fenwick, A. E., Gribble, A. D., Ife, R. J., Stevens, N., Witherington, J., “Diastereoselective synthesis, activity and chiral stability of cyclic alkoxyketone inhibitors of cathepsin K,” BIOORG. MED. CHEM. LETT. 11:199-202 (2001b).
Kamolmatyakul, S., Chen, W., Li, Y. P., “Interferon-gamma down-regulates gene expression of cathepsin K in osteoclasts and inhibits osteoclast formation,” J. DENT. RES. 80:351-355 (2001).
Katunuma, N., Matsui, A., Inubushi, T., Murata, E., Kakegawa, H., Ohba, Y., Turk, D., Turk, V., Tada, Y., Asao, T., “Structure-based development of pyridoxal propionate derivatives as specific inhibitors of cathepsin K in vitro and in vivo,” BIOCHEM. BIOPHYS. RES. COMMUN. 267:850-854 (2000).
Katunuma, N., Matsui, A., Kakegawa, T., Murata, E., Asao, T., Ohba, Y., “Study of the functional share of lysosomal cathepsins by the development of specific inhibitors,” ADV. ENZYME REGUL. 39:247-260 (1999).
LaLonde, J. M., Zhao, B., Smith, W. W., Janson, C. A., DesJarlais, R. L., Tomaszek, T. A., Carr, T. J., Thompson, S. K., Oh, H. J., Yamashita, D. S., “Use of papain as a model for the structure-based design of cathepsin K inhibitors: crystal structures of two papain-inhibitor complexes demonstrate binding to S′-subsites,” J. MED. CHEM. 41:4567-4576 (1998).
Lark, M. W., Stroup, G. B., James, I. E., Dodds, R. A., Hwang, S. M., Blake, S. M., Lechowska, B. A., Hoffman, S. J., Smith, B. R., Kapadia, R., Liang, X., Erhard, K., Ru, Y., Dong, X., Marquis, R. W., Veber, D., Gowen, M., “A potent small molecule, nonpeptide inhibitor of Cathepsin K (SB 331750) prevents bone matrix resorption in the ovariectomized rat,” BONE 30(5):746-753 (2002).
Leung-Toung, R., Li, W., Tam, T F, Karimian, K., “Thiol-dependent enzymes and their inhibitors: a review,” CURR MED CHEM 9:979-1002 (2002).
Marquis, R. W., Ru, Y., LoCastro, S. M., Zeng, J., Yamashita, D. S., Oh, H. J., Erhard, K. F., Davis, L. D., Tomaszek, T. A., Tew, D., “Azepanone-based inhibitors of human and rat cathepsin K,” J. MED. CHEM. 44:1380-1395 (2001a).
Marquis, R. W., Ru, Y., Yamashita, D. S., Oh, H. J., Yen, J., Thompson, S. K., Carr, TlJ., Levy, M. A., Tomaszek, T. A., Ijames, C. F., “Potent dipeptidylketone inhibitors of the cysteine protease cathepsin K,” BIOORG. MED. CHEM. 7:581-588 (1999).
Marquis, R. W., Ru, Y., Zeng, J., Trout, R. E., LoCastro, S. M., Gribble, A. D., Witherington, J., Fenwick, A. E., Garnier, B., Tomaszek, T., “Cyclic ketone inhibitors of the cysteine protease cathepsin K,” J. MED. CHEM. 44:725-736 (2001b).
Marquis, R. W., Yamashita, D. S., Ru, Y., LoCastro, S. M., Oh, H. J., Erhard, K. F., DesJarlais, R. L., Head, J. S., Smith, W. W., Zhao, B., “Conformationally constrained 1,3-diamino ketones: a series of potent inhibitors of the cysteine protease cathepsin K,” J. MED. CHEM. 41:3563-3567 (1998).
Matsumoto, K., Mizoue, K., Kitamura, K., Tse, W. C., Huber, C. P., Ishida, T., “Structural basis of inhibition of cysteine proteases by E-64 and its derivatives,” BIOPOLYMERS 51:99-107 (1999).
McGrath, M. E., Klaus, J. L., Barnes, M. G., Bromme, D., “Crystal structure of human cathepsin K complexed with a potent inhibitor,” NAT. STRUCR. BIOL. 4:105-109 (1997).
Ortego, J., Escribano, J., Coca-Prados, M., “Gene expression of proteases and protease inhibitors in the human ciliary epithelium and ODM-2 cells,” EXP. EYE RES. 65:289-299 (1997).
Patil, A. D., Freyer, A. J., Carte, B., Taylor, P. B., Johnson, R. K., Faulkner, D. J.: Haploscleridamine, a novel tryptamine-derived alkaloid from a sponge of the order haplosclerida: an inhibitor of cathepsin K, J NAT PROD 65:628-9 (2002b).
Patil, A. D., Freyer, A. J., Kilimer, L., Offen, P., Taylor, P. B., Votta, R. J., Johnson, R. K.: A new dimeric dihydrochalcone and a new prenylated flavone from the bud covers of Artocarpus altilis: potent inhibitors of cathepsin K, J NAT PROD 65:624-7 (2002a).
Percival, M. D., Ouellet, M., Campagnolo, C., Claveau, D., Li, C., “Inhibition of cathepsin K by nitric oxide donors: evidence for the formation of mixed disulfides and a sulfenic acid,” BIOCHEMISTRY 38: 13574-13583 (1999).
Schick, C., Pemberton, P. A., Shi, G. P., Kamachi, Y., Cataltepe, S., Bartuski, A. J., Gornstein, E. R., Bromme, D., Chapman, H. A., Silverman, G. A., “Cross-class inhibition of the cysteine proteinases cathepsins K, L, and S by the serpin squamous cell carcinoma antigen 1: a kinetic analysis,” BIOCHEMISTRY 37:5258-5266 (1998).
Shepard, A. R., Jacobson, N., Fingert, J. H., Stone, E. M., Sheffield, V. C., Clark, A. F., “Delayed secondary glucocorticoid responsiveness of MYOC in human trabecular meshwork cells,” IOVS 42:3173 (2001)
Smith, R. A., Bhargava, A., Browe, C., Chen, J., Dumas, J., Hatoum-Mokdad, H., Romero, R.: Discovery and parallel synthesis of a new class of cathepsin K inhibitors, BIOORG MED CHEM LETT 11:2951-4 (2001).
Steely, H. T., Browder, S. L., Julian, M. B., Miggans, S. T. Wilson, K. L., Clark, A. F., “The effects of dexamethasone on fibronectin expression in cultured human trabecular meshwork cells,” IOVS 33:2242 (1992).
Stroup, G. B., Lark, M. W., Veber, D. F., Bhattacharyya, A., Blake, S., Dare, L. C., Erhard, K. F., Hoffman, S. J., James, I. E., Marquis, R. W.: Potent and selective inhibition of human cathepsin K leads to inhibition of bone resorption in vivo in a nonhuman primate, J BONE MINER RES 16(10): 1739-46 (2001).
Thompson, S. K., Halbert, S. M., Bossard, M. J., Tomaszek, T. A., Levy, M. A., Zhao, B., Smith, W. W., Abdel-Meguid, S. S., Janson, C. A., D'Alessio, K. J., “Design of potent and selective human cathepsin K inhibitors that span the active site,” PROC. NATL. ACAD. SCI. U.S.A. 94:14249-14254 (1997).
Thompson, S. W, Halbert, S. M., DesJarlais, R. L., Tomaszek, T. A., Levy, M. A., Tew, D. G., Ijames, C. F., Veber, D. F., “Structure-based design of non-peptide, carbohydrazide-based cathepsin K inhibitors,” BIOORG. MED. CHEM. 7:599-605 (1999).
Thompson, S. K., Smith, W. W., Zhao, B., Halbert, S. M., Tomaxzek, T. A., Tew, D. G., Levy, M. A., Janson, C. A., KJ, D. A., McQueney, M. S., “Structure-based design of cathepsin K inhibitors containing a benzyloxy-substituted benzoyl peptidomimetic,” J. MED. CHEM. 41:3923-3927 (1998).
Turk, B., Turk, V., Turk, D., “Structural and functional aspects of papain-like cysteine proteinases and their protein inhibitors,” BIOL. CHEM. 378:141-150 (1997).
Votta, B. J., Levy, M. A., Badger, A., Bradbeer, J., Dodds, R. A., James, I. E., Thompson, S., Bossard, M. J., Carr, T., Connor, J. R., “Peptide aldehyde inhibitors of cathepsin K inhibit bone resorption both in vitro and in vivo,” J. BONE MINER. RES. 12:1396-1406 (1997).
Wang, W. H., McNatt, L. G., Shepard, A. R., Jacobson, N., Nishimura, D. Y., Stone, E. M., Sheffield, V. C., Clark, A. F., “Optimal procedure for extracting RNA from human ocular tissues and expression profiling of the congenital glaucoma gene FOXC1 using quantitative RT-PCR,” MOL VIS 7:89 (2001).
Wilson, K., McCartney, M. D., Miggans, S. T., Clark, A. F., “Dexamethasone induced ultrastructural changes in cultured human trabecular meshwork cells,” CURR EYE RES 12:783 (1993).
Yamashita, D. S. and Dodds, R. A., “Cathepsin K and the design of inhibitors of cathepsin K,” CURR. PHARM. DES. 6:1-24 (2000).
Yamashita, D. S., Dong, X., Oh, H. J., Brook, C. S., Tomaszek, T. A., Szewczuk, L., Tew, D. G., Veber, D. F., “Solid-phase synthesis of a combinatorial array of 1,3-bis(acylamino)-2-butanones, inhibitors of the cysteine proteases cathepsins K and L,” J. COMB. CHEM. 1:207-215 (1999).
Zhao, B., Janson, C. A., Amegadzie, B. Y., D'Alessio, K., Griffin, C., Hanning, C. R., Jones, C., Kurdyla, J., McQueney, M., Qiu, X., “Crystal structure of human osteoclast cathepsin K complex with E-64,” NAT. STRUCT. BIOL. 4:109-111 (1997).

We claim:

1. A method for treating glaucoma, said method comprising administering to a patient in need thereof a therapeutically effective amount of a composition comprising at least one cathepsin K antagonist.

2. The method of claim 1, wherein the cathepsin K antagonist is selected from the group consisting of monensin, brefeldin A, tunicamycin and 1,3-bis(acylamino)-2-propanone derivatives, cycloaltilisin 6, cycloaltilisin 7, AC-3-1, AC-3-3, AC-5-1, haploscleridamine, 5-(2-morpholin-4-yl-thoxy)-benzofuran-2-carboxylic acid ((S)-3-methyl-1-[3-oxo-1-[2-(3-pyridin-2-yl-phenyl)-ethenoyl]-azepan-4-ylcarbanoyl)-butyl)-amide (SB-331750), SB-357114, peptidomimetic aminomethyl ketones, α,α′-diacylamino ketones, alkoxymethyl ketones, cyanamides, pyridoxal propionate derivatives (including Clik-164 and Clik-166), SB-290190, α-alkoxyketone derivatives, cyanamide derivatives, and N_{α}-acyl-α-amino acid-(arylaminoethyl)amides.

3. The method of claim 1, wherein the cathepsin K antagonist is an arylaminoethyl amide.

4. The method of claim 1, wherein the cathepsin K antagost is selected from the group consisting of:
(2S,1′S)-2-(benzyloxycarbonyl)amino-N-[1′-(2-carboxythiazol-4-yl)-3′-methylbutyl]-4-methylpentanamide;
(2S,1′S)-2-(benzyloxycarbonyl)amino-N-[1′-(2-carboxamidothiazol-4-yl)-3′-methylbutyl]-4-methylpentanamide;
(2S,1′S)-2-(benzyloxycarbonyl)amino-N-[1′-(2-carboethoxythiazol-4-yl)-3′-methylbutyl]-4-methylpentanamide;
(2S,1′S)-2-(benzyloxycarbonyl)amino-N-[1′-(2-cyanothiazol-4-yl)-3′-methylbutyl]-4-methylpentanamide;
(2S,1′S)-2-(benzyloxycarbonyl)amino-N-[1′-[2-(N-benzylcarboxamido)thiazol-4-yl]-3′-methylbutyl]-4-methylpentanamide;
(2S,1′S)-2-(benzyloxycarbonyl)amino-N-[1-[2-[N′-3-methylpropyl)carboxamido]thiazol-4-yl)]-3′-methylbutyl]-4-methylpentanamide;
(2S,1′S)-2-(benzyloxycarbonyl)amino-N-[1′-[2-[N′-2-phenylethyl) carboxamido]thiazol-4-yl)]-3′-methylbutyl]-4-methylpentanamide;
(2S,1′S)-2-(benzyloxycarbonyl)amino-N-[1′-(4-carboethoxythiazol-2-yl)-3′-methylbutyl]-4-methylpentanamide;
(2S,1′S)-2-(benzyloxycarbonyl)amino-N-[1′-(4-carboxythiazol-2-yl)-3′-methylbutyl]4-methylpentanamide;
(2S,1′S)-2-(benzyloxycarbonyl)amino-N-[1′-(4-carboethoxythiadiazol-2-yl)-3′-methylbutyl]-4-methylpentanamide;
(2S,1′S)-2-(benzyloxycarbonyl)amino-N-[1′-(2-carbo-2,2,2-trifluoroethoxythiazol-4-yl)-3′-methylbutyl]-4-methylpentanamide;
(2S,1′S)-2-(benzyloxycarbonyl)amino-N-[1′-(4-carboethoxyoxadiazol-2-yl)-3′-methylbutyl]4-methylpentanamide;
(2S,1′S)-2-(benzyloxycarbonyl-L-leucinyl)amino-N-[1′-(4-carboethoxythiazol-2-yl)-3′-methylbutyl]-4-methylpentanamide;
(2S,1′S)-2-(benzyloxycarbonyl)amino-N-[1′-(4-carboxamidooxadiazol-2-yl)-3′-methylbutyl]-4-methylpentanamide;
(2S,1′S)-2-(benzyloxycarbonyl)amino-N-[1′-(2-carboethoxythiazol-4-yl)-3′-methylbutyl]-3-phenylpropanamide;
(2S,1′S)-2-(benzyloxycarbonyl-L-leucinyl)amino-N-[1′-(2-carboethoxythiazol-4-yl)-3′-methylbutyl]-4-methylpentanamide;
(2S,1′S)-2-(benzyloxycarbonyl)amino-N-[1′-(5-mercapto-1,2,4-oxadiazol-3-yl)-3′-methylbutyl]methylpentanamide;
(2S,1′S)-2-(benzyloxycarbonyl)amino-N-[1′-(2-mercaptothiazol-4-yl)-3′-methylbutyl]-4-methylpentanamide;
(2S)-2-(benzyloxycarbonyl)amino-N-(4-carboethoxythiazol-2-yl)methyl-4-methyl pentanamide;
(2S,1′S)-2-(benzyloxycarbonyl)amino-N-[1′-(2-benzyloxycarbonylthiazol-4-yl)-3′-methylbutyl]-4-methylpentanamide;
(2S,1′S)-2-(benzyloxycarbonyl)amino-4-methyl-N-[3′-methyl-1′-(2-phenoxycarbonylthiazol-4-yl)butyl]pentanamide;
(2S,1′S)-2-(benzyloxycarbonyl)amino-4-methyl-N-[3′-methyl-1′-[2-(2-methylpropyloxy carbonyl)thiazol-4-yl]butyl]pentanamide;
(2R,1′S)-2-(benzyloxycarbonyl)amino-N-[1′-(carboethoxythiazol-2-yl)ethyl]-4-methylpentanamide;
(2R,1′R)-2-(benzyloxycarbonyl)amino-N-[1′-(4-carbethoxythiazol-2-yl)ethyl]-4-methylpentanamide;
(2S,1′S)-N-[1′-(2-aminothiazol-4-yl)-3′-methylbutyl]-2-(benzyloxycarbonyl)amino-4-methylpentanamide;
(2S,1′S)-2-(benzyloxycarbonyl)amino-N-[1′-(2-carboethoxythiazol-4-yl)-3′-methylbutyl]-4-methylpentanamide;
(2S,1′S)-2-(benzyloxycarbonyl)amino-N-[1′-(4-carboethoxythiazol-2-yl)-3′-methylbutyl]-4-methylpentanamide;
(2S,1′S)-2-(benzyloxycarbonyl-L-leucinyl)amino-N-[1′-(4-carboethoxythiazol-2-yl)-3′-methylbutyl]-4-methylpentanamide;
(1S)-N-[4-[(1-benzyloxycarbonylamino)-3-methylbutyl]thiazol-2-ylcarbonyl]-N′-(N-benzyloxycarbonyl-L-leucinyl)hydrazide;
N-benzyloxycarbonyl-L-leucinyl-N′-benzyloxycarbonyl-L-leucinyl-L-leucinylhydrazide;
(1S)-N-[2-[(1-benzyloxycarbonylamino)-3-methylbutyl]thiazol-4-ylcarbonyl]-N′-(N-benzyloxycarbonyl-L-leucinyl)hydrazide;
2,2′-(N,N′-bis-benzyloxycarbonyl-L-leucinyl)carbohydrazide;
2,2′-(N,N′-bis-cyclohexylacetyl)carbohydrazide;
2,2′-(N,N′-bis-4-methylpentanoyl)carbohydrazide;
2,2′-(N,N′-bis-cyclopentylacetyl)carbohydrazide;
2,2′-(N,N′-bis-benzyloxycarbonylglycinyl)carbohydrazide;
2,2′-(N,N′-bis-acetyl-L-leucinyl)carbohydrazide;
2,2′-(N,N′-bis-benzyloxycarbonyl-L-alanyl)carbohydrazide;
2-(N-benzyloxycarbonyl-L-leucinyl)-2′-[N′-(4-methylpentanoyl)]carbohydrazide;
2,2′-(N,N′-bis-benzyloxycarbonyl-L-leucinyl)carbohydrazide;
bis-(Cbz-leucinyl)-1,3-diamino-propan-2-one;
bis-1,3-(4-phenoxy-benzoyl)-diamino-propan-2-one;
1-(Cbz-leucinyl)-amino-3-(acetyl-leucinyl)-amino-propan-2-one;
1-(Cbz-leucinyl)-amino-3-(Cbz-glutamyl-t-butyl ester)-amino-propan-2-one;
1-(Cbz-leucinyl)-amino-3-(Cbz-glutamyl)-amino-propan-2-one;
bis-1,3-(Cbz-leucinyl)-diamino-(S)-butanone-2-one;
1-(Cbz-leucinyl)-amino-3-(Cbz-phenylalanyl)-amino-propan-2-one;
1-(Cbz-leucinyl)-amino-3-(Cbz-norleucinyl)-amino-propan-2-one;
1-(Cbz-leucinyl)-amino-3-(Cbz-norvalinyl)-amino-propan-2-one;
bis-1,3-(Cbz-leucinyl)-diamino-5-methyl-(S)-hexan-2-one;
1-(acetyl-leucinyl)-amino-3-(4-phenoxy-benzoyl)-amino-propan-2-one;
1-(Cbz-homo-leucinyl)-amino-(Cbz-leucinyl)-3-amino-propan-2-one;
1-(Cbz-leucinyl)-amino-3-(acetyl-leucinyl)-amino-propan-2-one;
bis-1,3-(4-(3-chloro-2-cyano-phenoxy)-phenyl sulfonamido)-propan-2-one;
bis-1,3-(4-phenoxy-phenyl sulfonamido)-propan-2-one;
1-(Cbz-leucinyl)-amino-3-(4-(3-chloro-2-cyano-phenoxy)-phenyl sulfonamido)-propan-2-one;
1-(Cbz-leucinyl)-amino-3-(tosyl-amino)-propan-2-one;
1-(Cbz-leucinyl)-amino-3-((4-phenoxy-phenyl)-sulfonamido)-propan-2-one;
1-(Cbz-leucinyl)-amino-3-(2-dibenzofuransulfonamido)-propan-2-one;
1-(Cbz-homo-leucinyl)-amino-3-(2-dibenzofuransulfonamido)-propan-2-one;
1-(Cbz-leucinyl)-amino-3-(2-dibenzofuransulfonamido)-(S)-butan-2-one;
1-(Cbz-leucinyl)-amino-3-((4-phenoxy-phenyl)-sulfonamido)-propan-2-one;
1-(Cbz-leucinyl)-amino-3-(2-benzofuransulfonamido)-propan-2-one;
1-(Cbz-leucinyl)-amino-3-(2-dibenzofuransulfonamido)-(S)-butan-2-one;
(S)-Phenylmethyl [1-[[[3-[benzyloxycarbonyl-leucinyl-amino]-2-oxopropyl]-1-(benzyl)amino]carbonyl]-3-methylbutyl]carbamate;
(S)-Phenylmethyl [1-[[[3-[(2-dibenzofuranylsulfonyl)amino]-2-oxopropyl]-3-(benzyl)amino]carbonyl]-3-methylbutyl]carbamate;
(S)-Phenylmethyl [1-[[[3-[(2-benzofuranylsulfonyl)amino]-2-oxopropyl]-3-(4-pyridinylmethyl)amino]carbonyl]-3-methylbutyl]carbamate;
1-[[3-[(2-dibenzofuranylsulfonyl)amino]-2-oxopropyl]-3-(4-pyridinylmethyl)] benzamide;
(S)-Phenylmethyl [1-[[[3-[(2-dibenzofuranylsulfonyl)amino]-2-oxopropyl]-1-(4-pyridinylmethyl)amino]carbonyl]-3-methylbutyl]carbamate;
(S)-Phenylmethyl [1-[[[3-[(2-dibenzofuranylsulfonyl)amino]-2-oxopropyl]-1-(4-pyridinylmethyl)amino]carbonyl]-3-methylbutyl]carbamate;
2-[N-(N-benzyloxycarbonyl-L-leucinyl)]-2′-[N-(4-phenoxyphenylsulfonyl)]carbohydrazide;
2-[N-(N-benzyloxycarbonyl-L-alanyl)]-2′-[N-(N-benzyloxycarbonyl-L-leucinyl)] carbohydrazide;
2-[N-(N-benzyloxycarbonyl-L-leucinyl)]-2′-[N′-(4-phenylbenzoyl)]carbohydrazide;
2-[N-(N-benzyloxycarbonyl-L-leucinyl)]-2′-[N′-4-methoxybenzoyl)]carbohydrazide;
2-[N-(N-benzyloxycarbonyl-L-leucinyl)]-2′-[N′-(4-phenoxybenzoyl)]carbohydrazide;
2-(N-acetyl)-2′-[N′-(N-benzyloxycarbonyl-L-leucinyl)]carbohydrazide;
2-[N-(N-acetyl-L-leucinyl)]-2′-[N′-(N-benzyloxycarbonyl-L-alanyl)]carbohydrazide;
2-[N-(N-acetyl-L-alanyl)]-2′-[N′-(N-benzyloxycarbonyl-L-leucinyl)]carbohydrazide;
2-[N-(N-benzyloxycarbonyl-L-leucinyl)]-2′-[N′-[4-(N,N-dimethylaminomethyl) benzoyl)]]carbohydrazide;
2-[N-(N-benzyloxycarbonyl-L-leucinyl)]-2′-[N′-[4-hydroxy-[3-(4-morpholinome thyl)]]benzoyl]carbohydrazide;
2-[N-(N-benzyloxycarbonyl-L-leucinyl)]-2′-[N′-[4-[(N,N-dimethylaminomethyl) benzyloxy]carbonyl-L-leucinyl]carbohydrazide;
2-(N-benzoyl)-2′-[N′-(N-benzyloxycarbonyl-L-leucinyl)]carbohydrazide;
2-[N-(N-benzyloxycarbonyl-L-leucinyl)]-2′-[N′-[3-(4-morpholinomethyl)benzoyl]]carbohydrazide;
2-[N-(3-benzyloxybenzoyl)]-2′-[N′-(N-benzyloxycarbonyl-L-leucinyl)]carbohydrazide;
2-[N-(N-benzyloxycarbonyl-L-leucinyl)]-2′-[N′-[4-[3-N—N-dimethylamino)-1-propyloxy] benzoyl]]carbohydrazide;
2-[N-(2-benzyloxybenzoyl)]-2′-[N′-(N-benzyloxycarbonyl-L-leucinyl)]carbohydrazide;
2-[N-(N-benzyloxycarbonyl-L-leucinyl)]-2′-[N′-[3-(4-pyridylmethoxy)benzoyl]]carbohydrazide;
2-[N-(4-benzyloxybenzoyl)]-2′-[N′-(N-benzyloxycarbonyl-L-leucinyl)]carbohydrazide;
2-[N-(N-benzyloxycarbonyl-L-leucinyl)]-2′-[N′-(3-benzyloxy-5-methoxy)benzoyl] carbohydrazide;
2-[N-(N-benzyloxycarbonyl-L-leucinyl)]-2′-[N′-(3-benzyloxy-5-dimethoxy) benzoyl]carbohydrazide;
2-[N-(N-benzyloxycarbonyl-L-leucinyl)]-2′-[N′-(3-benzyloxy-5-ethoxy) benzoyl]carbohydrazide;
2-[N-(N-benzyloxycarbonylglycinyl)]-2′-[N′-(N-benzyloxycarbonyl-L-leucinyl)] carbohydrazide;
2-[N-(3-benzyloxybenzoyl)]-2′-[N′-(N-benzyloxycarbonyl-L-prolinyl)]carbohydrazide;
2-[N-(N-benzyloxycarbonyl-L-leucinyl)]-2′-[N′-(4-phenylphenylacetyl)]carbohydrazide;
(2′S)-2-[N-(3-benzyloxybenzoyl)]-2′-[N′-N-benzyloxycarbonyl-2-aminobutyryl)]carbohydrazide;
2,2′-[N,N′-[bis-4-phenylphenylacetyl)]]carbohydrazide;
(2′RS)-2-[N-(N-benzyloxycarbonyl-L-leucinyl)]-2′-[2-(4-phenylphenoxy)propionyl]carbohydrazide;
2-[N-(3-benzyloxybenzoyl)]-2′-[N′-(4-methylpentanoyl)]carbohydrazide;
(2RS,2′RS)-2,2′-[N,N′-[bis-[2-(4-phenylphenyl)-4-methylpentanoyl)]]]carbohydrazide;
(2′RS)-2-[N-(N-benzyloxycarbonyl-L-leucinyl)]-2′-[N′-[2-(4-phenylphenyl)-4-methylpentanoyl)]]carbohydrazide;
(2′RS)-2-[N-(3-benzyloxybenzoyl)]-2′-[N-[2-(4-phenylphenyl)-4-methylpentanoyl)]]carbohydrazide;
2-[N-(3-benzyloxybenzoyl)]-2′-[N′-(N-benzyloxycarbonyl-N-methyl-L-leucinyl)]carbohydrazide;
2-[N-(3-benzyloxybenzoyl)]-2′-[N′-[N-(2-pyridinylmethoxycarbonyl)-L-leucinyl]]carbohydrazide;
2-[N-[3-(4-pyridylmethoxy)benzoyl]]-2′-[N′-[N-(2-pyridinylmethoxycarbonyl)-L-leucinyl]]carbohydrazide;
(2RS)-2-[N-[2-(4-phenylphenyl)-4-methylpentanoyl)]]-2′-[N′-[N-(2-pyridinylmethoxycarbonyl)-L-leucinyl]]carbohydrazide;
2-[N-(N-benzyloxycarbonyl-L-leucinyl)]-2′-[N′-[2-(4-phenylphenyl)-4-methylpentanoyl)]]carbohydrazide;
2-[N-(N-benzyloxycarbonyl-L-leucinyl)]-2′-[N′-[2-(4-phenylphenyl)-4-methylpentanoyl)]]carbohydrazide;
2-[N-(N-benzyloxycarbonyl-L-leucinyl)]-2′-[N′-[N-(4-phenylphenyl)-N-(2-methylpropyl)carbamoyl]]carbohydrazide;
2-[N-(3-benzyloxybenzoyl)]-2′-[N′-(N-methyl-L-leucinyl)]carbohydrazide;
2-[N-(N-benzyloxycarbonyl-L-leucinyl)]-2′-[N′-(N-methyl-L-leucinyl)]carbohydrazide;
(1S)-N-[2-[(1-benzyloxycarbonylamino)-3-methylbutyl]thiazol-4-ylcarbonyl]-N′-(4-phenoxyphenylsulfonyl)hydrazide;
(1S)-N-[4-[1-(N-benzyloxycarbonyl-L-leucinylamino)-3-methylbutyl]thiazol-2-ylcarbonyl]-N′-(N-benzyloxycarbonyl-L-leucinyl)hydrazide;
(1S)-N-[2-[(1-benzyloxycarbonylamino)-3-methylbutyl]thiazol-4-ylcarbonyl]-N′-(4-phenylphenylacetyl)hydrazide;
(1S)-N-[2-[(1-benzyloxycarbonylamino)-3-methylbutyl]thiazol-4-ylcarbonyl]-N′-[3-(4-pryidinylmethoxy)benzoyl]hydrazide;
N-[2-(2-chlorophenoxymethyl)thiazol-4-ylcarbonyl]-N′-[N-(4-pyridinylmethoxycarbonyl)-L-leucinyl]hydrazide;
N-[N-(4-pyridinylmethoxycarbonyl)-L-leucinyl]-N′-[2-[4-(1,2,3-thiadiazol-4-yl)phenyl]thiazol-4-ylcarbonyl]hydrazide;
N-[2-[3-(4-chlorophenylsulfonylmethyl)thien-2-yl]thiazol-4-ylcarbonyl]-N′-[N-4-pyridinylmethoxycarbonyl)-L-leucinyl]hydrazide;
(1S,2′RS)-N-[2-[(1-benzyloxycarbonylamino)-3-methylbutyl]thiazol-4-ylcarbonyl]-N′-[2′-(4-phenylphenylacetyl)-4-methylpentanoyl-]hydrazide;
N-[2-(3-benzyloxyphenyl)thiazol-4-ylcarbonyl]-N′-[N-(2-pyridinylmethoxycarbonyl)-L-leucinyl]hydrazide;
(1RS)-N-[2-[1-(4-phenylphenyl)-3-methylbutyl]thiazol-4-ylcarbonyl]-N′-[N-(4-pyridinylmethoxycarbonyl)-L-leucinyl]hydrazide;
N-[2-(2-benzyloxyphenyl)thiazol-4-ylcarbonyl]-N′-[N-(4-pyridinylmethoxycarbonyl)-L-leucinyl]hydrazide;
N-[2-[N-methyl-N-(4-phenylphenyl)amino]thiazol-4-ylcarbonyl]-N′-[N-(4-pyridinyl methoxycarbonyl)-L-leucinyl]hydrazide;
N-(N-benzyloxycarbonyl-L-leucinyl)-N′-[2-(4-phenylbenzyl)thiazol-4-ylcarbonyl]hydrazide;
N-[2-(4-phenylphenylbenzyl)thiazol-4-ylcarbonyl]-N-[N-(4-pyridinylmethoxycarbonyl)-L-leucinyl]hydrazide;
N-(N-benzyloxycarbonyl-L-leucinyl)-N′-[2-[N-(2-methylpropyl)-N-phenylamino]thiazol-4-ylcarbonyl]hydrazide;
N-[2-[N-(2-methylpropyl)-N-phenylamino]thiazol-4-ylcarbonyl]-N′-[N-(4-pyridinyl methoxycarbonyl)-L-leucinyl]hydrazide;
N-[2-(2-benzyloxyphenyl)thiazol-4-ylcarbonyl]-N′-[N-(3-pyridinylmethoxycarbonyl)-L-leucinyl]hydrazide;
N-[2-(2-benzyloxyphenyl)thiazol-4-ylcarbonyl]-N′-[N-(2-pyridinylmethoxycarbonyl)-L-leucinyl]hydrazide;
N-(N-benzyloxycarbonyl-N-methyl-L-leucinyl)-N′-[2-(2-benzyloxyphenyl)thiazol-4-ylcarbonyl]hydrazide;
N-[2-[N-(2-methylpropyl)-N-phenylamino]thiazol-4-ylcarbonyl]-N′-[N-(2-pyridinylmethoxycarbonyl)-L-leucinyl]hydrazide;
N-[2-[N-(2-methylpropyl)-N-phenylamino]thiazol-4-ylcarbonyl]-N′-[N-(3-pyridinyl methoxycarbonyl)-L-leucinyl]hydrazide;
N-[2-(2-methoxyphenyl)thiazol-4-ylcarbonyl]-N′-[N-(4-pyridinylmethoxycarbonyl)-L-leucinyl]hydrazide;
2-[N-(N-benzyloxycarbonyl-L-leucinyl)]-2′-[N′-[4-(N,N-dimethylaminomethyl) benzyloxy]carbonyl-L-leucinyl]carbonhydrazide;
(3S,4S)-3-(2S-2-benzyloxycarbonylamino-2-cyclohexyl-methyl-acetamido)-4-acetoxy-azetidin-2-one;
(3S,4S)-3-{2S-2-(3-phenylpropionoyl)amino-2-cyclo-hexylmethyl-acetamido}-4-acetoxy-azetidin-2-one;
(3S,4S)-3-{2S-2-(3-phenylpropionoyl)amino-2-cyclo-hexylmethyl-acetamido}-4-{4-(2S-2-amino-2-carboxy-ethyl)-phenoxy}-azetidin-2-one;
(3S,4R)-3-{2S-2-(3-phenylpropionoyl)amino-2-cyclo-hexylmethyl-acetamido}4-{4-(2S-2-amino-2-carboxy-ethyl)-phenoxy}-azetidin-2-one;
(3S,4SR)-3-{2S-2-(3-phenylpropionoyl)amino-2-cyclo-hexylmethyl-acetamido}-4-phenylthio-azetidin-2-one;
(3S,4SR)-3-{2S-2-(3-phenylpropionoyl)amino-2-cyclo-hexylmethyl-acetamido}-4-phenylsulfonyl-azetidin-2-one;
(3S,4S)-3-{2S-2-(benzylaminocarbonyl)amino-2-cyclo-hexylmethyl-acetamido}-4-acetoxy-azetidin-2-one;
(3S,4S)-3-{2S-2-(phenylethenylsulfonyl)amino-2-cyclohexylmethyl-acetamido}-4-acetoxy-azetidin-2-one;
(3S,4S)-3-(2S-2-benzyloxycarbonylamino-2-cyclohexylmethyl-acetamido)-4-(3-methyl-phenoxy)-azetidin-2-one;
(3S,4R)-3-(2S-2-benzyloxycarbonyl amino-2-cyclohexylmethyl-acetamido)-4-(3-methyl-phenoxy)-azetidin-2-one;
(3S,4S)-3-{2S-2-[3-(pyridin-4-yl) propenoyl]amino-2-cyclohexylmethyl-acetamido}4-phenoxy-azetidin-2-one; and
(3S,4S)-3-{2S-2-[3-(pyridin-3-yl)propenoyl]amino-2-cyclohexylmethyl-acetamido}4-phenoxy-azetidin-2-one.

5. A composition for the treatment of glaucoma, said composition comprising a therapeutically effective amount of at least one cathepsin K antagonist and a pharmaceutically acceptable excipient.

6. The composition of claim 5, wherein the cathepsin K antagonist is selected from the group consisting of arylaminoethyl amides, monensin, brefeldin A, tunicamycin and 1,3-bis(acylamino)-2-propanone derivatives, cycloaltilisin 6, cycloaltilisin 7, AC-3-1, AC-3-3, AC-5-1, haploscleridamine, 5-(2-morpholin-4-yl-thoxy)-benzofuran-2-carboxylic acid ((S)-3-methyl-1-[3-oxo-1-[2-(3-pyridin-2-yl-phenyl)-ethenoyl]-azepan-4-ylcarbanoyl)-butyl)-amide (SB-331750), SB-357114, peptidomimetic aminomethyl ketones, α,α′-diacylamino ketones, alkoxymethyl ketones, cyanamides, pyridoxal propionate derivatives (including Clik-164 and Clik-166), SB-290190, α-alkoxyketone derivatives, cyanamide derivatives, and N_{α}-acyl-α-amino acid-(arylaminoethyl)amides.

7. The composition of claim 5, wherein the cathepsin K antagonist is an arylaminoethyl amide.

8. The composition of claim 5, wherein the cathepsin K antagonist is selected from the group consisting of:
(2S,1′S)-2-(benzyloxycarbonyl)amino-N-[1′-(2-carboxythiazol-4-yl)-3′-methylbutyl]-4-methylpentanamide;
(2S,1′S)-2-(benzyloxycarbonyl)amino-N-[1′-(2-carboxamidothiazol-4-yl)-3′-methylbutyl]-4-methylpentanamide;
(2S,1′S)-2-(benzyloxycarbonyl)amino-N-[1′-(2-carboethoxythiazol-4-yl)-3′-methylbutyl]-4-methylpentanamide;
(2S,1′S)-2-(benzyloxycarbonyl)amino-N-[′-(2-cyanothiazol-4-yl)-3′-methylbutyl]-4-methylpentanamide;
(2S,1′S)-2-(benzyloxycarbonyl)amino-N-[1′-[2-(N′-benzylcarboxamido)thiazol-4-yl]-3′-methylbutyl]4-methylpentanamide;
(2S,1′S)-2-(benzyloxycarbonyl)amino-N-[1′-[2-[N′-3-methylpropyl)carboxamido]thiazol-4-yl)]-3′-methylbutyl]-4-methylpentanamide;
(2S,1′S)-2-(benzyloxycarbonyl)amino-N-[1′-[2-[N′-2-phenylethyl) carboxanmido]thiazol-4-yl)]-3′-methylbutyl]-4-methylpentanamide;
(2S,1′S)-2-(benzyloxycarbonyl)amino-N-[1′-(4-carboethoxythiazol-2-yl)-3′-methylbutyl]4-methylpentanamide;
(2S,l′S)-2-(benzyloxycarbonyl)amino-N-[1′-(4-carboxythiazol-2-yl)-3′-methylbutyl]-4-methylpentanamide;
(2S,1′S)-2-(benzyloxycarbonyl)amino-N-[1′-(4-carboethoxythiadiazol-2-yl)-3′-methylbutyl]-4-methylpentanamide;
(2S,1′S)-2-(benzyloxycarbonyl)amino-N-[1′-(2-carbo-2,2,2-trifluoroethoxythiazol-4-yl)-3′-methylbutyl]-4-methylpentanamide;
(2S,1′S)-2-(benzyloxycarbonyl)amino-N-[1′-(4-carboethoxyoxadiazol-2-yl)-3′-methylbutyl]4-methylpentanamide;
(2S,1′S)-2-(benzyloxycarbonyl-L-leucinyl)amino-N-[1′-(4-carboethoxythiazol-2-yl)-3′-methylbutyl]-4-methylpentanamide;
(2S,1′S)-2-(benzyloxycarbonyl)amino-N-[1′-(4-carboxamidooxadiazol-2-yl)-3′-methylbutyl]-4-methylpentanamide;
(2S,1′S)-2-(benzyloxycarbonyl)amino-N-[1′-(2-carboethoxythiazol-4-yl)-3′-methylbutyl]-3-phenylpropanamide;
(2S,1′S)-2-(benzyloxycarbonyl-L-leucinyl)amino-N-[1′-(2-carboethoxythiazol-4-yl)-3′-methylbutyl]-4-methylpentanamide;
(2S,1′S)-2-(benzyloxycarbonyl)amino-N-[1′-(5-mercapto-1,2,4-oxadiazol-3-yl)-3′-methylbutyl]-4-methylpentanamide;
(2S,1′S)-2-(benzyloxycarbonyl)amino-N-[1′-(2-mercaptothiazol-4-yl)-3′-methylbutyl]-4-methylpentanamide;
(2S)-2-(benzyloxycarbonyl)amino-N-(4-carboethoxythiazol-2-yl)methyl-4-methyl pentanamide;
(2S,1′S)-2-(benzyloxycarbonyl)amino-N-[1′-(2-benzyloxycarbonylthiazol-4-yl)-3′-methylbutyl]-4-methylpentanamide;
(2S,1′S)-2-(benzyloxycarbonyl)amino-4-methyl-N-[3′-methyl-1′-(2-phenoxycarbonylthiazol-4-yl)butyl]pentanamide;
(2S,1′S)-2-(benzyloxycarbonyl)amino-4-methyl-N-[3′-methyl-1′-[2-(2-methylpropyloxy carbonyl)thiazol-4-yl]butyl]pentanamide;
(2R,1′S)-2-(benzyloxycarbonyl)amino-N-[1′-(carboethoxythiazol-2-yl)ethyl]-4-methylpentanamide;
(2R,1′R)-2-(benzyloxycarbonyl)amino-N-[1′-(4-carbethoxythiazol-2-yl)ethyl]-4-methylpentanamide;
(2S,1′S)-N-[1′-(2-aminothiazol-4-yl)-3′-methylbutyl]-2-(benzyloxycarbonyl)amino-4-methylpentanamide;
(2S,1′S)-2-(benzyloxycarbonyl)amino-N-[1′-(2-carboethoxythiazol-4-yl)-3′-methylbutyl]-4-methylpentanamide;
(2S,1′S)-2-(benzyloxycarbonyl)amino-N-[1′-(4-carboethoxythiazol-2-yl)-3′-me thylbutyl]4-methylpentanamide;
2S,1′S)-2-(benzyloxycarbonyl-L-leucinyl)amino-N-[1′-(4-carboethoxythiazol-2-yl)-3′-methylbutyl]-4-methylpentanamide;
(1S)-N-[4-[(1-benzyloxycarbonylamino)-3-methylbutyl]thiazol-2-ylcarbonyl]-N′-(N-benzyloxycarbonyl-L-leucinyl)hydrazide;
N-benzyloxycarbonyl-L-leucinyl-N′-benzyloxycarbonyl-L-leucinyl-L-leucinylhydrazide;
(1S)-N-[2-[(1-benzyloxycarbonylamino)-3-methylbutyl]thiazol-4-ylcarbonyl]-N′-(N-benzyloxycarbonyl-L-leucinyl)hydrazide;
2,2′-(N,N′-bis-benzyloxycarbonyl-L-leucinyl)carbohydrazide;
2,2′-(N,N′-bis-cyclohexylacetyl)carbohydrazide;
2,2′-(N,N′-bis-4-methylpentanoyl)carbohydrazide;
2,2′-(N,N-bis-cyclopentylacetyl)carbohydrazide;
2,2′-(N,N′-bis-benzyloxycarbonylglycinyl)carbohydrazide;
2,2′-(N,N′-bis-acetyl-L-leucinyl)carbohydrazide;
2,2′-(N,N′-bis-benzyloxycarbonyl-L-alanyl)carbohydrazide;
2-(N-benzyloxycarbonyl-L-leucinyl)-2′-[N′-(4-methylpentanoyl)]carbohydrazide;
2,2′-(N,N′-bis-benzyloxycarbonyl-L-leucinyl)carbohydrazide;
bis-(Cbz-leucinyl)-1,3-diamino-propan-2-one;
bis-1,3-(4-phenoxy-benzoyl)-diamino-propan-2-one;
1-(Cbz-leucinyl)-amino-3-(acetyl-leucinyl)-amino-propan-2-one;
1-(Cbz-leucinyl)-amino-3-(Cbz-glutamyl-t-butyl ester)-amino-propan-2-one;
1-(Cbz-leucinyl)-amino-3-(Cbz-glutamyl)-amino-propan-2-one;
bis-1,3-(Cbz-leucinyl)-diamino-(S)-butanone-2-one;
1-(Cbz-leucinyl)-amino-3-(Cbz-phenylalanyl)-amino-propan-2-one;
1-(Cbz-leucinyl)-amino-3-(Cbz-norleucinyl)-amino-propan-2-one;
1-(Cbz-leucinyl)-amino-3-(Cbz-norvalinyl)-amino-propan-2-one;
bis-1,3-(Cbz-leucinyl)-diamino-5-methyl-(S)-hexan-2-one;
1-(acetyl-leucinyl)-amino-3-(4-phenoxy-benzoyl)-amino-propan-2-one;
1-(Cbz-homo-leucinyl)-amino-(Cbz-leucinyl)-3-amino-propan-2-one;
1-(Cbz-leucinyl)-amino-3-(acetyl-leucinyl)-amino-propan-2-one;
bis-1,3-(4-(3-chloro-2-cyano-phenoxy)-phenyl sulfonamido)-propan-2-one;
bis-1,3-(4-phenoxy-phenyl sulfonamido)-propan-2-one;
1-(Cbz-leucinyl)-amino-3-(4-(3-chloro-2-cyano-phenoxy)-phenyl sulfonamido)-propan-2-one;
1-(Cbz-leucinyl)-amino-3-(tosyl-amino)-propan-2-one;
1-(Cbz-leucinyl)-amino-3-((4-phenoxy-phenyl)-sulfonamido)-propan-2-one;
1-(Cbz-leucinyl)-amino-3-(2-dibenzofuransulfonamido)-propan-2-one;
1-(Cbz-homo-leucinyl)-amino-3-(2-dibenzofuransulfonamido)-propan-2-one;
1-(Cbz-leucinyl)-amino-3-(2-dibenzofuransulfonamido)-(S)-butan-2-one;
1-(Cbz-leucinyl)-amino-3-((4-phenoxy-phenyl)-sulfonamido)-propan-2-one;
1-(Cbz-leucinyl)-amino-3-(2-dibenzofuransulfonamido)-propan-2-one;
1-(Cbz-leucinyl)-amino-3-(2-dibenzofuransulfonamido)-(S)-butan-2-one;
(S)-Phenylmethyl [1-[[[3-[benzyloxycarbonyl-leucinyl-amino]-2-oxopropyl]-1-(benzyl)amino]carbonyl]-3-methylbutyl]carbamate;
(S)-Phenylmethyl [1-[[[3-[(2-dibenzofuranylsulfonyl)amino]-2-oxopropyl]-3-(benzyl)amino]carbonyl]-3-methylbutyl]carbamate;
(S)-Phenylmethyl [1-[[[3-[(2-dibenzofuranylsulfonyl)amino]-2-oxopropyl]-3-(4-pyridinylmethyl)amino]carbonyl]-3-methylbutyl]carbamate;
1-[[3-[(2-dibenzofuranylsulfonyl)amino]-2-oxopropyl]-3-(4-pyridinylmethyl)] benzamide;
(S)-Phenylmethyl [1-[[[3-[(2-dibenzofuranylsulfonyl)amino]-2-oxopropyl]-1-(4-pyridinylmethyl)amino]carbonyl]-3-methylbutyl]carbamate;
(S)-Phenylmethyl [1-[[[3-[(2-dibenzofuranylsulfonyl)amino]-2-oxopropyl]-1-(4-pyridinylmethyl)amino]carbonyl]-3-methylbutyl]carbamate;
2-[N-(N-benzyloxycarbonyl-L-leucinyl)]-2′-[N′-(4-phenoxyphenylsulfonyl)]carbohydrazide;
2-[N-(N-benzyloxycarbonyl-L-alanyl)]-2′-[N-(N-benzyloxycarbonyl-L-leucinyl)]carbohydrazide;
2-[N-(N-benzyloxycarbonyl-L-leucinyl)]-2′-[N′-(4-phenylbenzoyl)]carbohydrazide;
2-[N-(N-benzyloxycarbonyl-L-leucinyl)]-2′-[N′-4-methoxybenzoyl)]carbohydrazide;
2-[N-N-benzyloxycarbonyl-L-leucinyl)]-2′-[N′-(4-phenoxybenzoyl)]carbohydrazide;
2-(N-acetyl)-2‘-N’-(N-benzyloxycarbonyl-L-leucinyl)]carbohydrazide;
2-[N-(N-acetyl-L-leucinyl)]-2′-[N′-(N-benzyloxycarbonyl-L-alanyl)]carbohydrazide;
2-[N-(N-acetyl-L-alanyl)]-2′-[N′-(N-benzyloxycarbonyl-L-leucinyl)]carbohydrazide;
2-[N-(N-benzyloxycarbonyl-L-leucinyl)]-2′-[N′-[4-(N,N-dimethylaminomethyl) benzoyl)]]carbohydrazide;
2-[N-(N-benzyloxycarbonyl-L-leucinyl)]-2′-[N′-[4-hydroxy-[3-(4-morpholinome thyl)]]benzoyl]carbohydrazide;
2-[N-(N-benzyloxycarbonyl-L-leucinyl)]-2′-[N′-[4-[(N,N-dimethylaminomethyl) benzyloxy]carbonyl-L-leucinyl]carbohydrazide;
2-(N-benzoyl)-2′-[N′-(N-benzyloxycarbonyl-L-leucinyl)]carbohydrazide;
2-[N-(N-benzyloxycarbonyl-L-leucinyl)]-2′-[N′-[3-(4-morpholinomethyl)benzoyl]]carbohydrazide;
2-[N-(3-benzyloxybenzoyl)]-2′-[N′-(N-benzyloxycarbonyl)-L-leucinyl)]carbohydrazide;
2-[N-(N-benzyloxycarbonyl-L-leucinyl)]-2′-[N′-[4-[3-N—N-dimethylamino)-1-propyloxy]benzoyl]]carbohydrazide;
2-[N-(2-benzyloxybenzoyl)]-2′-[N′-(N-benzyloxycarbonyl-L-leucinyl)]carbohydrazide;
2-[N-(N-benzyloxycarbonyl-L-leucinyl)]-2′-[N′-[3-(4-pyridylmethoxy)benzoyl]]carbohydrazide;
2-[N-(4-benzyloxybenzoyl)]-2′-[N′-(N-benzyloxycarbonyl-L-leucinyl)]carbohydrazide;
2-[N-(N-benzyloxycarbonyl-L-leucinyl)]-2′-[N′-(3-benzyloxy-5-methoxy)benzoyl] carbohydrazide;
2-[N-(N-benzyloxycarbonyl-L-leucinyl)]-2′-[N′-(3-benzyloxy-4,5-dimethoxy) benzoyl]carbohydrazide;
2-[N-(N-benzyloxycarbonyl-L-leucinyl)]-2′-[N′-(3-benzyloxy-5-ethoxy) benzoyl]carbohydrazide;
2-[N-(N-benzyloxycarbonylglycinyl)]-2′-[N′-(N-benzyloxycarbonyl-L-leucinyl)]carbohydrazide;
2-[N-(3-benzyloxybenzoyl)]-2′-[N′-(N-benzyloxycarbonyl-L-prolinyl)]carbohydrazide;
2-[N-N-benzyloxycarbonyl-L-leucinyl)]-2′-[N′-(4-phenylphenylacetyl)]carbohydrazide;
(2′S)-2-[N-(3-benzyloxybenzoyl)]-2′-[N′-(N-benzyloxycarbonyl-2-aminobutyryl)]carbohydrazide;
2,2′-[N,N-[bis-4-phenylphenylacetyl)]]carbohydrazide;
(2′RS)-2-[N-(N-benzyloxycarbonyl-L-leucinyl)]-2′-[2-(4-phenylphenoxy)propionyl] carbohydrazide;
2-[N-(3-benzyloxybenzoyl)]-2′-[N′-(4-methylpentanoyl)]carbohydrazide;
(2RS,2′RS)-2,2′-[N,N′-[bis-[2-(4-phenylphenyl)-4-methylpentanoyl)]]]carbohydrazide;
(2′RS)-2-[N-N-benzyloxycarbonyl-L-leucinyl)]-2′-[N′-[2-(4-phenylphenyl)-4-methylpentanoyl)]]carbohydrazide;
(2′RS)-2-[N-(3-benzyloxybenzoyl)]-2′-[N-[2-(4-phenylphenyl)-4-methylpentanoyl)]] carbohydrazide;
2-[N-(3-benzyloxybenzoyl)]-2′-[N′-(N-benzyloxycarbonyl-N-methyl-L-leucinyl)] carbohydrazide;
2-[N-(3-benzyloxybenzoyl)]-2′-[N′-[N-(2-pyridinylmethoxycarbonyl)-L-leucinyl]]carbohydrazide;
2-[N-[3-(4-pyridylmethoxy)benzoyl]]-2′-[N′-[N-(2-pyridinylmethoxycarbonyl)-L-leucinyl]]carbohydrazide;
(2RS)-2-[N-[2-(4-phenylphenyl)-4-methylpentanoyl)]]-2′-[N′-[N-(2-pyridinylmethoxy carbonyl)-L-leucinyl]]carbohydrazide;
2-[N-(N-benzyloxycarbonyl-L-leucinyl)]-2′-[′-[2-(4-phenylphenyl)-4-methylpentanoyl)]] carbohydrazide;
2-[N-(N-benzyloxycarbonyl-L-leucinyl)]-2′-[′-[2-(4-phenylphenyl)-4-methylpentanoyl)]] carbohydrazide;
2-[N-(N-benzyloxycarbonyl-L-leucinyl)]-2′-[N′-[N-(4-phenylphenyl)-N-(2-methylpropyl) carbamoyl]]carbohydrazide;
2-[N-(3-benzyloxybenzoyl)]-2′-[N′-(N-methyl-L-leucinyl)]carbohydrazide;
2-[N-(N-benzyloxycarbonyl-L-leucinyl)]-2′-[N′-(N-methyl-L-leucinyl)]carbohydrazide;
(1S)-N-[2-[(1-benzyloxycarbonylamino)-3-methylbutyl]thiazol-4-ylcarbonyl]-N′-(4-phenoxyphenylsulfonyl)hydrazide;
(1S)-N-[4-[1-(N-benzyloxycarbonyl-L-leucinylamino)-3-methylbutyl]thiazol-2-ylcarbonyl]-N′-(N-benzyloxycarbonyl-L-leucinyl)hydrazide;
(1S)-N-[2-[(1-benzyloxycarbonylamino)-3-methylbutyl]thiazol-4-ylcarbonyl]-N′-(4-phenylphenylacetyl)hydrazide;
(1S)-N-[2-[(1-benzyloxycarbonylamino)-3-methylbutyl]thiazol-4-ylcarbonyl]-N′-[3-(4-pryidinylmethoxy)benzoyl]hydrazide;
N-[2-(2-chlorophenoxymethyl)thiazol-4-ylcarbonyl]-N′-[N-(4-pyridinylmethoxycarbonyl)-L-leucinyl]hydrazide;
N-[N-(4-pyridinylmethoxycarbonyl)-L-leucinyl]-N′-[2-[4-(1,2,3-thiadiazol-4-yl)phenyl]thiazol-4-ylcarbonyl]hydrazide;
N-[2-[3-(4-chlorophenylsulfonylmethyl)thien-2-yl]thiazol-4-ylcarbonyl]-N′-[N-(4-pyridinylmethoxycarbonyl)-L-leucinyl]hydrazide;
(1S,2′RS)-N-[2-[(1-benzyloxycarbonylamino)-3-methylbutyl]thiazol-4-ylcarbonyl]-N′-[2′-(4-phenylphenylacetyl)-4-methylpentanoyl-]hydrazide;
N-[2-(3-benzyloxyphenyl)thiazol-4-ylcarbonyl]-N′-[N-(2-pyridinylmethoxycarbonyl)-L-leucinyl]hydrazide;
(1RS)-N-[2-[1-(4-phenylphenyl)-3-methylbutyl]thiazol-4-ylcarbonyl]-N′-[N-(4-pyridinylmethoxycarbonyl)-L-leucinyl]hydrazide;
N-[2-(2-benzyloxyphenyl)thiazol-4-ylcarbonyl]-N′-[N-(4-pyridinylmethoxycarbonyl)-L-leucinyl]hydrazide;
N-[2-[N-methyl-N-(4-phenylphenyl)amino]thiazol-4-ylcarbonyl]-N′-[N-(4-pyridinyl methoxycarbonyl)-L-leucinyl]hydrazide;
N-(N-benzyloxycarbonyl-L-leucinyl)-N′-[2-(4-phenylbenzyl)thiazol-4-ylcarbonyl]hydrazide;
N-[2-(4-phenylphenylbenzyl)thiazol-4-ylcarbonyl]-N′-[N-(4-pyridinylmethoxycarbonyl)-L-leucinyl]hydrazide;
N-(N-benzyloxycarbonyl-L-leucinyl)-N′-[2-[N-(2-methylpropyl)-N-phenylamino]thiazol-4-ylcarbonyl]hydrazide;
N-[2-[N-(2-methylpropyl)-N-phenylamino]thiazol-4-ylcarbonyl]-N-[N-(4-pyridinyl methoxycarbonyl)-L-leucinyl]hydrazide;
N-[2-(2-benzyloxyphenyl)thiazol-4-ylcarbonyl]-N′-[N-(3-pyridinylmethoxycarbonyl)-L-leucinyl]hydrazide;
N-[2-(2-benzyloxyphenyl)thiazol-4-ylcarbonyl]-N′-[N-(2-pyridinylmethoxycarbonyl)-L-leucinyl]hydrazide;
N-(N-benzyloxycarbonyl-N-methyl-L-leucinyl)-N′-[2-(2-benzyloxyphenyl)thiazol-4-ylcarbonyl]hydrazide;
N-[2-[N-(2-methylpropyl)-N-phenYlamino]thiazol-4-ylcarbonyl]-N′-[N-(2-pyridinyl methoxycarbonyl)-L-leucinyl]hydrazide;
N-[2-[N-(2-methylpropyl)-N-phenylamino]thiazol-4-ylcarbonyl]-N′-[N-(3-pyridinyl methoxycarbonyl)-L-leucinyl]hydrazide;
N-[2-(2-methoxyphenyl)thiazol-4-ylcarbonyl]-N′-[N-(4-pyridinylmethoxycarbonyl)-L-leucinyl]hydrazide;
2-[N-(N-benzyloxycarbonyl-L-leucinyl)]-2′-[N′-[4-(N,N-dimethylaminomethyl) benzyloxy]carbonyl-L-leucinyl]carbonhydrazide;
(3S,4S)-3-(2S-2-benzyloxycarbonylamino-2-cyclohexyl-methyl-acetamido)-4-acetoxy-azetidin-2-one;
(3S,4S)-3-{2S-2-(3-phenylpropionoyl)amino-2-cyclo-hexylmethyl-acetamido}-4-acetoxy-azetidin-2-one;
(3S,4S)-3-{2S-2-(3-phenylpropionoyl)amino-2-cyclo-hexylmethyl-acetamido}-4-{4-(2S-2-amino-2-carboxy-ethyl)-phenoxy}-azetidin-2-one;
(3S,4R)-3-{2S-2-(3-phenylpropionoyl)amino-2-cyclo-hexylmethyl-acetamido}-4-{4-(2S-2-amino-2-carboxy-ethyl)-phenoxy}-azetidin-2-one;
(3S,4SR)-3-{2S-2-(3-phenylpropionoyl)amino-2-cyclo-hexylmethyl-acetamido}-4-phenylthio-azetidin-2-one;
(3S,4SR)-3-{2S-2-(3-phenylpropionoyl)amino-2-cyclo-hexylmethyl-acetamido}-4-phenylsulfonyl-azetidin-2-one;
(3S,4S)-3-{2S-2-(benzylaminocarbonyl)amino-2-cyclo-hexylmethyl-acetamido}-4-acetoxy-azetidin-2-one;
(3S,4S)-3-{2S-2-(phenylethenylsulfonyl)amino-2-cyclohexylmethyl-acetamido}-4-acetoxy-azetidin-2-one;
(3S,4S)-3-(2S-2-benzyloxycarbonylamino-2-cyclohexylmethyl-acetamido)-4-(3-methyl-phenoxy)-azetidin-2-one;
(3S,4R)-3-(2S-2-benzyloxycarbonyl amino-2-cyclohexylmethyl-acetamido)-4-(3-methyl-phenoxy)-azetidin-2-one;
(3S,4S)-3-{2S-2-[3-pyridin-4-yl) propenoyl]amino-2-cyclohexylmethyl-acetamido}-4-phenoxy-azetidin-2-one; and
(3S,4S)-3-{2S-2-[3-(pyridin-3-yl) propenoyl]amino-2-cyclohexylmethyl-acetamido}4-phenoxy-azetidin-2-one.
